SEQUENCE LISTING (3 sequences)

SEQ ID NO: 1 - protein, 420 aa, synthetic construct
KFPIYTIPDKLGPWSPIDIMHLSCPNNLVVEDEGCTNLSGFSYMELKVGYISAIKMNGFTCTGVVTEAETYTNFVGYVTTTFKRKHFRPTPDACRAAYNWKMAGDPRYEESLHNPYPDYHWLRTVKTTKESLVIISPSVADLDPYDRSLHSRVFPGGNCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPGQLVNLQDFRSDEIEHLVVEELVKKREECLDALELIMTTKSVSFRRLSHLRKLVPGFGKAYTIFNKTLMEADAHYKSVRTWNEIIPSKGCLRVGGRCHPHVNGVFFNGIILGPDGNVLIPEMQSSLLQQHMELLVSSVIPLMHPLADPSTVFKNGDEAEDFVEVHL

SEQ ID NO: 2 - protein, 420 aa, synthetic construct
KFPIYTIPDKLGPWSPIDIMHLSCPNNLVVEDEGCTNLSGFSYMELKVGYISAIKMNGFTCTGVVTEAETYTNFVGAVTTTFKRKHFRPTPDACRAAYNWKMAGDPRYEESLHNPYPDYHWLRTVKTTKESLVIISPSVADLDPYDRSLHSRVFPGGNCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPGQLVNLQDFRSDEIEHLVVEELVKKREECLDALELIMTTKSVSFRRLSHLRKLVPGFGKAYTIFNKTLMEADAHYKSVRTWNEIIPSKGCLRVGGRCHPHVNGVFFNGIILGPDGNVLIPEMQSSLLQQHMELLVSSVIPLMHPLADPSTVFKNGDEAEDFVEVHL

SEQ ID NO: 3 - protein, 420 aa, Synthetic construct
KFPIYTIPDKLGPWSPIDIHHLSCPNNLVVEDEGCTNLSGFSYMELKVGYISAIKMNGFTCTGVVTEAETYTNFVGYVTTTFKRKHFRPTPDACRAAYNWKMAGDPRYEESLHNPYPDYHWLRTVKTTKESLVIISPSVADLDPYDRSLHSRVFPGGNCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPGQLVNLHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGNVLIPEMQSSLLQQHMELLVSSVIPLMHPLADPSTVFKNGDEAEDFVEVHL

(19) United States
(12) Patent Application Publication
(10) Pub. No.: US 2024/0425550 A1
(43) Pub. Date: Dec. 26, 2024

(54) ENGINEERED RABIES VIRUS GLYCOPROTEIN, COMPOSITIONS, AND METHODS OF USE THEREOF

(71) Applicant: LA JOLLA INSTITUTE FOR IMMUNOLOGY, La Jolla, CA (US)

(72) Inventors: Erica Ollmann Saphire, La Jolla, CA (US); Kathryn Hastie, La Jolla, CA (US); Heather Callaway, La Jolla, CA (US)

(21) Appl. No.: 18/700,922

(22) PCT Filed: Oct. 14, 2022
(86) PCT No.: PCT/US2022/078162

§ 371 Date: Apr. 12, 2024

Related U.S. Application Data
(60) Provisional application No. 63/256,383, filed on Oct. 15, 2021.
(60) Provisional application No. 63/398,291, filed on Aug. 16, 2022.

(51) Int. Cl. C07K 14/005; A61K 39/00; A61P 31/14

(52) U.S. Cl. CPC C07K 14/005; A61P 31/14; A61K 39/00; C07K 2319/70; C12N 2760/20122; C12N 2760/20134

(57) ABSTRACT

Provided herein are, inter alia, methods and compositions for treating and preventing rhabdoviridae infection, including rabies virus. Compositions include recombinant rabies virus glycoproteins that are able to form glycoprotein trimers. The glycoprotein trimers are contemplated to be effective for preventing and/or treating rabies virus infections, including for use in the formulation of rabies virus vaccine compositions.

### CROSS-REFERENCE TO RELATED APPLICATIONS

This application claims the benefit of priority to U.S. Provisional Application No. 63/256,383, filed Oct. 15, 2021 and U.S. Provisional Application No. 63/398,291, filed Aug. 16, 2022, both of which are incorporated by reference in their entirety.

### REFERENCE TO SEQUENCE LISTING

This application incorporates by reference a Computer Readable Form (CRF) of a Sequence Listing in ASCII text format submitted with this application, entitled 048513-516001WO_SL.xml, was created on Oct. 13, 2022, and is 4,839 bytes in size.

### BACKGROUND

Rabies virus can infect both humans and all other mammalian species, and is the causative agent of rabies. Rabies is nearly 100% lethal if untreated and causes over 50,000 human deaths every year, mostly in children. In spite of vaccines and anti-viral antibodies, rabies virus remains a threat to global human and animal health, largely because existing rabies vaccines do not elicit long-term immune responses in most vaccines. In animals, annual booster vaccines are required to maintain protection, which can create a large financial burden for domestic animal owners and the livestock industry. In humans, rabies vaccines are usually only administered after a potential rabies exposure, and must be delivered in multiple doses and alongside an expensive antiviral antibody cocktail derived from human sera in order to be effective. In low income countries, this treatment is often either unavailable or prohibitively expensive, and rabies infection results in death.

Rabies vaccines usually consist of inactivated rabies virus and elicit an antibody response against rabies virus glycoprotein (RabvG), the only protein exposed on the surface of rabies virus. RabvG adopts a variety of conformations, readily transitioning between its pre-fusion and post-fusion conformations and between monomeric and trimeric states. The heterogeneity of RabvG likely prevents the immune system from generating a long-lasting immune response to rabies virus after vaccination.

Therefore, generation of a RabvG that mimics the native pre-fusion GP trimer is an essential point for vaccine design. Disclosed herein, inter alia, are solutions to these and other problems in the art.

### BRIEF SUMMARY OF THE INVENTION

In an aspect is provided a recombinant rabies virus glycoprotein or variant, homologue, derivative or subsequence thereof.

In an aspect is provided a recombinant rabies virus glycoprotein.

In an aspect is provided a recombinant rabies virus glycoprotein, comprising one or more of the following mutations: His20Met, His261Gln, Tyr77Ala, and Ser289Leu.

In an aspect is provided a recombinant rabies virus glycoprotein, having an amino acid sequence of SEQ ID NO:1 or SEQ ID NO:2.

In an aspect is provided a recombinant rabies virus glycoprotein or variant, homologue, derivative or subsequence thereof, having an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:1 or SEQ ID NO: 2.

In an aspect is provided a recombinant rabies virus glycoprotein, having an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:1 or SEQ ID NO:2.

In another aspect is provided a glycoprotein trimer including the recombinant rabies virus glycoproteins provided herein including embodiments thereof, wherein the recombinant rabies virus glycoproteins are bound by non-covalent attachment of trimerization domains.

In an aspect is provided a nucleic acid encoding a recombinant rabies virus glycoprotein provided herein including embodiments thereof.

In another aspect a cell is provided, the cell including a recombinant rabies virus glycoprotein provided herein including embodiments thereof or the glycoprotein trimer provided herein including embodiments thereof.

In an aspect is provided a cell including a nucleic acid provided herein including embodiments thereof.

In an aspect is provided a vaccine composition including the recombinant rabies virus glycoprotein provided herein including embodiments thereof and a pharmaceutically acceptable excipient.

In another aspect is provided a vaccine composition including the glycoprotein trimer provided herein including embodiments thereof and a pharmaceutically acceptable excipient.

In an aspect a method of treating or preventing a viral disease in a subject in need of such treatment or prevention is provided, the method including administering a therapeutically or prophylactically effective amount of a recombinant rabies virus glycoprotein provided herein including embodiments thereof to the subject.

In an aspect a method of treating or preventing a viral disease in a subject in need of such treatment or prevention is provided, the method including administering a therapeutically or prophylactically effective amount of a glycoprotein trimer provided herein including embodiments thereof to the subject.

In an aspect a method for immunizing a subject susceptible to a viral disease is provided, the method including administering a recombinant rabies virus glycoprotein provided herein including embodiments thereof to the subject under conditions such that antibodies directed to the rabies virus glycoprotein or a fragment thereof are produced.

In an aspect a method for immunizing a subject susceptible to a viral disease is provided, the method including administering a glycoprotein trimer provided herein including embodiments thereof to the subject under conditions such that antibodies directed to the glycoprotein trimer or a fragment thereof are produced.

In an aspect is provided a method of diagnosing rabies virus infection in a subject, the method including: (a) contacting a biological sample obtained from the subject with the recombinant rabies virus glycoprotein provided herein including embodiments thereof, and (b) detecting binding of one or more antibodies to said recombinant rabies virus glycoprotein, thereby diagnosing rabies virus infection in said subject.

In an aspect is provided a method of diagnosing rabies virus infection in a subject, the method including: (a) contacting a biological sample obtained from the subject with the glycoprotein trimer provided herein including embodiments thereof, and (b) detecting binding of one or more antibodies to said glycoprotein trimer, thereby diagnosing rabies virus infection in said subject.

In an aspect is provided a method for evaluating effectiveness of a rabies virus vaccine in a subject, comprising (a) contacting a biological sample from a subject who has been administered with a vaccine for a rabies virus with an engineered immunogen described herein, (b) detecting antibodies in the biological sample that specifically bind to the engineered immunogen, and (c) performing quantitative and qualitative analysis of the antibodies detected in the biological sample, thereby evaluating effectiveness of the rabies virus vaccine in the subject.

In an aspect is provided an antibody directed to a recombinant rabies virus glycoprotein provided herein including embodiments thereof. In an aspect is provided an antibody directed to a glycoprotein trimer provided herein including embodiments thereof.

In an aspect a method of generating rabies virus-specific antibodies is provided, the method including administering any of the compositions provided herein including embodiments thereof to a subject, obtaining biological material from the subject, and purifying antibodies from the biological material.

In an aspect a method for detecting rabies virus infection is provided, the method including contacting a biological sample with an antibody provided herein, and detecting the presence or absence of rabies virus.

A method of determining the presence of antibodies specific for a rabies virus in a biological sample including contacting the biological sample with a composition including a recombinant rabies virus glycoprotein provided herein, and detecting the presence or absence of rabies virus-specific antibody.

In an aspect, provided herein is use of a recombinant rabies virus glycoprotein as provided herein including embodiments thereof or a glycoprotein trimer as provided herein for the manufacture of a medicament for the treatment in a subject of a viral disease.

In an aspect, provided herein is a recombinant rabies virus glycoprotein as provided herein including embodiments thereof or a glycoprotein trimer as provided herein for use in the treatment of a viral disease in a subject in need thereof.

### BRIEF DESCRIPTION OF THE DRAWINGS

FIGS. 1A-1D provides a non-limiting example of the structure of a pre-fusion, trimeric RabvG with stabilizing point mutations. Three protomers are shown from the top view (1A) and side view (1C) in shades of grey, with the relevant regions magnified (1B and 1D) and stabilizing mutations highlighted.

FIG. 2 provides a non-limiting example showing that stabilizing mutations 289L and 20M/261Q/289L decrease the pH at which RabvG fuses cells. 293T cells constitutively expressing eGFP or mCherry were seeded together in 24 well plates and transfected to express full-length RabvG. 2 days post transfection, cells were incubated in 0.1M citric acid/trisodium citrate buffer at the indicated pH or left in DMEM growth medium (No Change) for 10 minutes at room temperature, then washed with PBS and incubated with DMEM for 1 hr at 37° C. Cells were fixed and imaged to assess fusion. Brighter cells indicate the formation of syncytia (cell fusion). Wild-type RabvG fused cells at pH 5.83. The 289L mutant fused cells at pH 5.17 and the 20M/261Q/289L mutant did not fuse at any of the tested pHs. This indicates that the 289L mutant increases protein stability and the 20M/261Q/289L mutant greatly increases protein stability.

FIG. 3 provides a non-limiting example showing that 20M, 261Q, 289L, and 20M/261Q/289L full-length RabvG proteins are not shed into tissue culture supernatant. 293T cells were transfected with full-length RabvG carrying the indicated point mutations. 4 days post-transfection, tissue culture supernatant was collected and an ELISA was performed to measure the amount of RabvG cleaved from the cell surface and released into the supernatant. Results were analyzed via ANOVA; **** p<0.0001. RabvG containing the point mutations 20M, 261Q, and 289L singly or in combination was not cleaved and released into tissue culture supernatant to significant levels.

FIGS. 4A and 4B provide non-limiting examples showing 20M, 261Q, 289L, and 20M/261Q/289L mutant full-length RabvG reach the cell surface to the same levels as wild-type RabvG. 293T cells were transfected to express full-length RabvG with the indicated point mutations. 2 days post transfection, cells were fixed and stained for analysis of surface-expressed RabvG via flow cytometry (4A) and IFA (4B). For flow cytometry experiments (4A), cells were gated on recognition by a polyclonal anti-RabvG antibody to detect transfected cells, and then recognition of these cells by a pre-fusion conformation specific monoclonal antibody was measured. The level of surface expressed RabvG was not significantly different between the wild-type protein or any of the point mutants. Additionally, there was no significant difference in the proportion of surface expressed RabvG in the pre-fusion conformation.

FIGS. 5A-5E provide a non-limiting example of the structure of the pre-fusion rabies virus glycoprotein trimer bound to neutralizing antibody RVA122. Molecular model of the rabies virus glycoprotein and RVA122 complex fitted into the corresponding electron density (5A) and magnified view of the fusion loops embedded in a liposome or micelle density (5B). (5C) Raw micrographs of glycoprotein complexes in interaction with micelles (top, single membrane) and liposomes (bottom, double membrane) and corresponding annotations to denote top views, side views, and liposome or micelle densities. (5D) and (5E) represent magnified portions of the micrographs of glycoprotein complexes in interaction with micelles.

FIGS. 6A and 6B provide a non-limiting example of the trimeric interface of the pre-fusion rabies virus glycoprotein and comparison in structures between trimeric RabvG, monomeric RabvG and VSV G. 6A) (Top) Book-end model of the interface between RabvG protomers, with contacts shown and the glycoprotein's central helix denoted with dotted lines. (Lower left) Magnified view of the interface between protomers, with bonds denoted by dotted lines. (Lower Right) Comparison between the central helices in the RabvG and VSV G trimeric structures, shown with distances between contacts (left) and as an overlay (right). 6B) Comparison between the fusion loops of the RabvG trimer, RabvG pre-fusion and post-fusion monomers, and the VSV G pre-fusion trimer. A conformationally heterogeneous region leading into the fusion loops is boxed.

FIGS. 7A-7E provide non-limiting example showing a quaternary binding epitope for neutralizing antibody RVA122. (7A): Binding of antibody RVA122 heavy chain (darkest grey, top-front in 7A, lower-center in 7B) and light chain (coupled with heavy chain, top-rear in 7A, upper-center in 7B) to two RabvG protomers as a side view (7B) (left) and top view (right, top). Binding footprint of RVA122 onto the RabvG protomers (right, bottom). (7D): Comparison of binding footprints for RVA122, antibody 523-11 and antibody xx. (7C): Molecular interactions between RVA122 and RabvG. (7E): Bio-layer interferometry measuring binding kinetics between RabvG and RVA122 antigen binding fragments (Fabs).

FIGS. 8A-8E provide anon-limiting example of the effects of fusion loop mutations on glycoprotein trimerization, binding kinetics, and conformation. (8A): Atomic model of the RabvG fusion loops. (8B): Crosslinking experiment measuring trimerization of wild-type and mutant RabvG ectodomains (His-tag). (8C): Bio-layer interferometry binding experiment measuring binding between combinations of wild-type RabvG ectodomains, F74A mutant RabvG ectodomains, and neutralizing antibody RVA122. (8D): ELISA measuring total secretion of soluble RabvG ectodomains (solid bars) and secreted RabvG in the pre-fusion conformation (striped bass). (8E): Negative stain EM reconstructions of wild-type F74A, and 74/77/119/121A mutant RabvG ectodomains (gray) that were purified via immunoprecipitation with neutralizing antibody RVA122 (highlighted). Error bars show SEM and asterisks denote: *=p<0.05.

FIGS. 9A-9E provide non-limiting examples of the effects of fusion loop mutations on full-length RabvG. 9A) Immunofluorescence assay on wild-type and mutant RabvG showing cell-surface expression of full-length RabvG, and pre-fusion conformation RabvG. 9B) Flow cytometry assay quantifying the level of pre-fusion conformation RabvG in transfected cells positive for (gated on) RabvG expression. 9C) Representative histogram (top) and bar graph displaying mean fluorescence intensity (MFI) (bottom). 9D) ELISAs measuring total amount of shed RabvG (left), shed RabvG in the pre-fusion conformation (middle) and the proportion of pre-fusion shed RabvG relative to total shed RabvG (right). 9E). Negative stain EM reconstructions of shed RabvG purified via immunoprecipitation with neutralizing antibody RVA122. Liposome densities are visible below wild-type, F74A, Y77A, and Y119A densities. The 74/77/119/121A combination mutant formed both monomeric and trimeric complexes, and the relative abundance of each population is shown. Error bars show SEM and asterisks denote: *=p≤0.05. **=p≤0.01.

FIGS. 10A-10D provide non-limiting examples of information and statistics for the cryo-EM structure of the rabies virus glycoprotein complexed with neutralizing antibody RVA122. A) Local resolution of the cryo-EM density map and B) 2D class averages showing representative top views, side views, and tilted views of particles included in the final particle set used for reconstruction. Fourier shell correlation (FSC) curve of the final cryo-EM map showing overall map resolution (C) and zoomed in view of the map and corresponding model to show a region of local resolution (D).

FIGS. 11A-11C provide non-limiting examples of a comparison between the RabvG pre-fusion trimeric structure and the pre-fusion monomeric structure. 11A) Structure of the pre-fusion monomer (top, PDB ID: 61gx), a protomer from the pre-fusion trimeric structure (center), and an overlay (bottom). 11B) Overlay of individual domains for the pre-fusion RabvG monomeric (gray) and trimeric structures (shaded by domain). 11C) Magnified overlay of Domain IV for the pre-fusion monomeric and trimeric structures.

FIGS. 12A-12D provide non-limiting examples of the quaternary binding interaction between neutralizing antibody RVA122 and RabvG. 12A and 12B) Side view showing interaction between RVA122 and two RabvG protomers, forming a quaternary binding interaction. 12C) Quaternary epitope-forming salt bridge between RabvG residue E269 and RVA122 residue R95. 12D) Binding kinetics between RabvG and R95E (left) and R95A (right) mutant RVA122.

FIGS. 13A-13D provide a non-limiting example of the expression of mutant RabvG ectodomains and additional crosslinking experiments. 13A)-13C) Crosslinking experiment measuring trimerization of wild-type and mutant RabvG ectodomains (double strep-tag). 13D) Western blot on total cell lysate, showing expression levels of wild-type and mutant soluble RabvG ectodomains.

FIG. 14A-14B provide a non-limiting example of negative stain electron micrographs of wild-type and mutant shed RabvG. Raw micrographs of negative stain electron micrographs for wild-type (14A) and 74/77/119/121A mutant (14B) shed RabvG used in negative stain reconstructions. Liposome densities co-purified with shed RabvG are visible for the wild-type but not for the 74/77/119/121A mutant.

FIGS. 15A-15F provide non-limiting examples of a structure pre-fusion, trimeric RabvG with stabilizing point mutations. Two adjacent protomers from the RABV-G trimer are shown from the side view (15A) and all three protomers are shown from the top view (15B). (15C-15F) are magnified views of RABV-G with indicated mutated residues.

FIG. 16 provides a non-limiting example of the effects of stabilizing mutations on RABV-G fusion.

FIG. 17 provides anon-limiting example of a negative stain electron microscopy of stabilized RABV-G ectodomains in solution and full-length stabilized RABV-G on pseudovirus. (17A) 3D reconstruction of 20M/77A/261Q/289L RABV-G ectodomains, with a wild-type RABV-G trimer docked in the electron density. Electron micrographs (17B) with particles used to reconstruct the 3D model and 2D classes (17C) are also shown. (17D) Negative stain electron micrograph of full-length 20M/261Q/289L RABV-G in vesicular stomatitis virus pseudoviruses.

FIGS. 18A-18E provide anon-limiting example of antibody titer and immune longevity for mice vaccinated with wild-type and stabilized (20M/261Q/289L) RABV-G. (18A) ELISA of serum from mice immunized with full-length RABV-G plasmids encoding WT or stabilized RABV-G. (18B-18E) Neutralization data for mouse serum collected at the indicated timepoint post-vaccination. Neutralization curves at 12-weeks (18B) and 20-weeks (C18) are shown, along with IC₅₀ values at 12-weeks (18D) and 20-weeks (18E).

### DETAILED DESCRIPTION

In an aspect, provided herein is a recombinant rabies virus protein. In an embodiment, the recombinant rabies virus protein is a glycoprotein. In embodiments, the oligomerization state of the recombinant rabies virus protein is a glycoprotein trimer. In embodiments, the recombinant rabies virus protein is a stabilized glycoprotein trimer.

In an aspect, provided herein is a recombinant rabies virus protein or peptide. In an embodiment, the recombinant rabies virus protein or peptide is a stabilized trimer.

In an aspect, provided herein is a recombinant rabies virus protein, peptide, variant, homologue, derivative, or subsequence thereof.

In an aspect, provided herein is a recombinant rabies virus protein or peptide comprising at least one mutation selected from:
(a) a methionine at an amino acid position corresponding to position 20 of SEQ ID NO:3;
(b) a glutamine at an amino acid position corresponding to position 261 of SEQ ID NO:3
(c) an alanine at an amino acid position corresponding to position 77 of SEQ ID NO:3; and,
(d) a serine at an amino acid position corresponding to position 289 of SEQ ID NO:3; or corresponding mutation in a variant, homologue, derivative, subsequence, or functional fragment thereof.

In an aspect, provided herein is a recombinant rabies virus protein or peptide comprising at least one mutation selected from:
(a) a methionine residue at an amino acid position corresponding to position 20 of SEQ ID NO:3;
(b) a glutamine residue at an amino acid position corresponding to position 261 of SEQ ID NO:3
(c) an alanine residue at an amino acid position corresponding to position 77 of SEQ ID NO:3; and,
(d) a serine residue at an amino acid position corresponding to position 289 of SEQ ID NO:3.

In an aspect, provided herein is a recombinant rabies virus protein having a sequence identity of at least 80% to the amino acid sequence as set forth in SEQ ID NO:3. In embodiments, the recombinant rabies virus protein has a sequence identity of at least 80% to the amino acid sequence as set forth in SEQ ID NO:3. In embodiments, the recombinant rabies virus protein has a sequence identity of at least 85% to the amino acid sequence as set forth in SEQ ID NO:3. In embodiments, the recombinant rabies virus protein has a sequence identity of at least 90% to the amino acid sequence as set forth in SEQ ID NO:3. In embodiments, the recombinant rabies virus protein has a sequence identity of at least 91% to the amino acid sequence as set forth in SEQ ID NO:3. In embodiments, the recombinant rabies virus protein has a sequence identity of at least 92% to the amino acid sequence as set forth in SEQ ID NO:3. In embodiments, the recombinant rabies virus protein has a sequence identity of at least 93% to the amino acid sequence as set forth in SEQ ID NO:3. In embodiments, the recombinant rabies virus protein has a sequence identity of at least 94% to the amino acid sequence as set forth in SEQ ID NO:3. In embodiments, the recombinant rabies virus protein has a sequence identity of at least 95% to the amino acid sequence as set forth in SEQ ID NO:3. In embodiments, the recombinant rabies virus protein has a sequence identity of at least 96% to the amino acid sequence as set forth in SEQ ID NO:3. In embodiments, the recombinant rabies virus protein has a sequence identity of at least 97% to the amino acid sequence as set forth in SEQ ID NO:3. In embodiments, the recombinant rabies virus protein has a sequence identity of at least 98% to the amino acid sequence as set forth in SEQ ID NO:3. In embodiments, the recombinant rabies virus protein has a sequence identity of at least 99% to the amino acid sequence as set forth in SEQ ID NO:3. In embodiments, the recombinant rabies virus protein has a sequence identity of at least 99.5% to the amino acid sequence as set forth in SEQ ID NO:3.

In an aspect, provided herein is a method of treating or preventing a viral disease in a subject comprising administering a therapeutically or prophylactically effective amount of the recombinant rabies virus protein or peptide, corresponding mutation in a variant, homologue, derivative, subsequence, or functional fragment thereof to the subject. In embodiments, the recombinant rabies virus has a sequence identity of at least 80% to the amino acid sequence as set forth in SEQ ID NO:3. In embodiments, the recombinant rabies virus protein has the amino acid sequence as set forth in SEQ ID NO: 1. In embodiments, the recombinant rabies virus protein has the amino acid sequence as set forth in SEQ ID NO:2. In embodiments, the viral disease is caused by a member of the Rhabdoviridae family. In embodiments, the viral disease is a lyssavirus. In embodiments, the viral disease is a rabies virus. In embodiments, the subject is a mammal. In a further embodiment, the mammal is a human, canine, cat, ferret, rabbit, cattle, horse, monkey, or coyote. In an embodiment, the mammal is a human. In another embodiment, the mammal is a canine, cat, ferret, rabbit, cattle, horse, monkey, or coyote.

The recombinant rabies virus glycoprotein provided herein (RabvG-R), including embodiments thereof, include a modified rabies virus glycoprotein ectodomain and, optionally, a trimerization domain. In certain embodiments, the rabies virus glycoproteins form glycoprotein trimers. In some embodiments, the glycoprotein trimers are formed by the rabies virus glycoproteins non-covalently binding each other through trimerization domains. The glycoprotein trimers are recognized by neutralizing antibodies. Thus, compositions including the recombinant rabies virus glycoprotein and or glycoprotein trimers are contemplated to be effective for treating and/or preventing rabies virus infection and associated diseases.

Compositions including the recombinant rabies virus glycoprotein and or glycoprotein trimers may further be used in antibody discovery. The compositions may be used as in diagnostic methods to characterize antibody response upon natural infection or vaccination. The compositions are further contemplated to be useful for characterizing the structure of rabies virus proteins and are useful for small molecule drug design targeting exogenous glycoprotein domains.

### Definitions

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

The use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc. References identified herein are expressly incorporated herein by reference in their entireties unless otherwise indicated.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

A “chemical linker,” as provided herein, is a covalent linker, a substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene or any combination thereof.

The chemical linker as provided herein may be a bond, —O—, —S—, —C(O)—, —C(O)O—, —C(O)NH—, —S(O)₂NH—, —NH—, —NHC(O)NH—, substituted (e.g., substituted with a substituent group, a size-limited substituent or a lower substituent group) or unsubstituted alkylene, substituted (e.g., substituted with a substituent group, a size-limited substituent or a lower substituent group) or unsubstituted heteroalkylene, substituted (e.g., substituted with a substituent group, a size-limited substituent or a lower substituent group) or unsubstituted cycloalkylene, substituted (e.g., substituted with a substituent group, a size-limited substituent or a lower substituent group) or unsubstituted heterocycloalkylene, substituted (e.g., substituted with a substituent group, a size-limited substituent or a lower substituent group) or unsubstituted arylene or substituted (e.g., substituted with a substituent group, a size-limited substituent or a lower substituent group) or unsubstituted heteroarylene.

The chemical linker as provided herein may be a bond, —O—, —S—, —C(O)—, —C(O)O—, —C(O)NH—, —S(O)₂NH—, —NH—, —NHC(O)NH—, substituted or unsubstituted (e.g., C₁-C₂₀, C₁-C₁₀, C₁-C₅) alkylene, substituted or unsubstituted (e.g., 2 to 20 membered, 2 to 10 membered, 2 to 5 membered) heteroalkylene, substituted or unsubstituted (e.g., C₃-C₈, C₃-C₆, C₃-C₅) cycloalkylene, substituted or unsubstituted (e.g., 3 to 8 membered, 3 to 6 membered, 3 to 5 membered) heterocycloalkylene, substituted or unsubstituted (e.g., C₆-C₁₀, C₆-C₈, C₆-C₅) arylene or substituted or unsubstituted (e.g., 5 to 10 membered, 5 to 8 membered, 5 to 6 membered) heteroarylene.

In embodiments, the chemical linker is a covalent linker. In embodiments, the chemical linker is a hydrocarbon linker.

Thus, a chemical linker as provided herein may include a plurality of chemical moieties, wherein each of the plurality of chemical moieties is chemically different. In embodiments, a chemical linker is formed using conjugate chemistry including, but not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition).

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different the general chemical structure of a nucleic acid, but that functions in a manner similar to a naturally occurring nucleic acid. Examples of such analogues include, without limitation, phosphorothioates, phosphoramidates, methyl phosphonates, chiral-methyl phosphonates, 2-O-methyl ribonucleotides, and peptide-nucleic acids (PNAs).

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In aspects, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

The term “complementary” or “complementarity” refers to the ability of a nucleic acid to form hydrogen bond(s) with another nucleic acid sequence by either traditional Watson-Crick or other non-traditional types. For example, the sequence A-G-T is complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions (i.e., stringent hybridization conditions).

The term “epitope” as used herein refers to the sequence or three-dimensional portion of the antigen that is recognized by antibodies. The recognized portion of the antigen may be the tertiary structure formed from the linear amino acid sequence. The three-dimensional portion of the antigen that is recognized by an antibody is also referred to as the conformational epitope. In embodiments, the antibodies are B cells or T cells.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., sgRNA) may be detected by standard PCR or Northern blot methods well known in the art. See, Sambrook et al., 1989 Molecular Cloning: A Laboratory Manual, 18.1-18.88.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window” as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length Win the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “rabies virus glycoprotein”, “RabvG”, or “RABV GP” as provided herein includes any of the recombinant or naturally-occurring forms of rabies virus glycoprotein (RabvG), also known as Pre-glycoprotein polyprotein GP complex, Pre-GP-C, or variants or homologs thereof that maintain RABV GP activity (e.g. within at least 50%, 80%, 90%, 95%, 96%, 97%, 98%, 99% or 100% activity compared to RABV GP). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring RABV GP protein polypeptide. In embodiments, RABV GP protein is the protein as identified by the UniProt reference number P16288, or a variant, homolog or functional fragment thereof. In aspects, the wild-type rabies virus glycoprotein includes the amino acid sequence of SEQ ID NO: 3. In aspects, the recombinant RABV GP includes the amino acid sequence of SEQ ID NO:1 or SEQ ID NO:2. In aspects, RABV GP has the amino acid sequence of SEQ ID NO: or SEQ ID NO:21. In aspects, RABV GP has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:3. In aspects, RABV GP includes an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:3

As used interchangeably herein, the terms “fusion protein” and “fusion polypeptide” refer to a polypeptide or an amino acid sequence linked to at least a second polypeptide or an amino acid sequence derived from a second polypeptide. The individualized elements of the fusion protein can be linked in any of a variety of ways, including for example, direct attachment, the use of an intermediate or spacer peptide, or the use of a linker region. In embodiments, the linker region is a covalent bond or a peptide linker. For example, the linker peptide includes anywhere from 0 to 100 amino acids, from 0 to 90 amino acids, from 0 to 80 amino acids, from 0 to 70 amino acids, from 0 to 60 amino acids, from 0 to 50 amino acids, from 0 to 55 amino acids, from 0 to 40 amino acids, from 0 to 35 amino acids, from 0 to 30 amino acids, from 0 to 25 amino acids, from 0 to 20 amino acids, from 0 to 15 amino acids, from 0 to 10 amino acids, 0 zero to 5 amino acids, or 0 zero to 3 amino acids. In embodiments, the polypeptide (e.g. GP1) is linked to a second polypeptide (e.g. GP2) by way of a covalent bond (e.g. a peptide bond). In embodiments, the polypeptide is linked to a second polypeptide by one or more disulfide linkages. For example, a polypeptide (e.g. GP1) may include a first cysteine amino acid side chain which may form a disulfide bond with a second cysteine amino acid side chain in a second polypeptide (e.g. GP2), thereby forming a fusion protein. Thus, in embodiments, the fusion protein includes two polypeptides (e.g. GP1 and GP2) covalently attached by way of one or more disulfide bonds. Thus, in embodiments, the fusion protein is a non-linear polypeptide.

The term “glycoprotein” or “GP” refers to proteins that include oligosaccharides covalently attached to amino acid side-chains. In embodiments, the GP is a Rabies virus (RABV) GP.

As used interchangeably herein, the terms “signal peptide” and “signal sequence” refer to a protein or peptide sequence that enables a cell to translocate a protein. In embodiments, the protein is translocated to the cell membrane.

The term “antigen” as used herein refers to the molecular structure that binds to a specific antibody or T-cell receptor. The presence of an antigen in a subject, such as a mammal, may elicit an immune a response. In embodiments, an antigen may be a protein, a polysaccharide, a lipid, a nucleic acid, a peptide or the like.

The term “dendritic cell” as used herein refers to a type of immune cell found in tissues of mammals and increases immune responses by presenting antigens on its surface to other cells, such as T-cells, of the immune system for recognition. In embodiments, a dendritic cell is a phagocyte. Dendritic cells are present in high density in mammalian tissue that is contact with the external environment such as skin, the inner lining of nose, lungs, stomach, and intestines.

The term “antigen-presenting cell” as used herein refers to a group of immune cells that mediate the cellular immune response via endocytosis and phagocytosis and presenting antigens for recognition by certain lymphocytes such as T-cells. The antigen is bound to the surface of the cell by major histocompatibility complex proteins. In embodiments, antigen-presenting cells include dendritic cells, macrophages, Langerhans cells, and B-cells.

The term “lymphocyte” as used herein refers to a type of immune cell that is made in the bone marrow and is found in the blood and in lymph tissue. In embodiments, lymphocytes include natural killer cells, T-cells, and B-cells. White blood cells include, granulocytes (neutrophils, eosinophils, and basophils), monocytes, and lymphocytes (T-cells and B-cells).

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions (also referred to herein as light chain variable (VL) domain and heavy chain variable (VH) domain, respectively) come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab₂, bispecific Fab₂, trispecific Fab₃, monovalent IgGs, scFv, bispecific antibodies, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”, “CDR H2” and “CDR H3” as provided herein refer to the complementarity determining regions (CDR) 1, 2, and 3 of the variable heavy (H) chain of an antibody. In embodiments, the variable heavy chain provided herein includes in N-terminal to C-terminal direction a CDR H1, a CDR H2 and a CDR H3.

The terms “FR L1”, “FR L2”, “FR L3” and “FR L4” as provided herein are used according to their common meaning in the art and refer to the framework regions (FR) 1, 2, 3 and 4 of the variable light (L) chain of an antibody. In embodiments, the variable light chain provided herein includes in N-terminal to C-terminal direction a FR L1, a FR L2, a FR L3 and a FR L4. Likewise, the terms “FR H1”, “FR H2”, “FR H3” and “FR H4” as provided herein are used according to their common meaning in the art and refer to the framework regions (FR) 1, 2, 3 and 4 of the variable heavy (H) chain of an antibody. In embodiments, the variable heavy chain provided herein includes in N-terminal to C-terminal direction a FR H1, a FR H2, a FR H3 and a FR H4.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL), variable light chain (VL) domain or light chain variable region and variable heavy chain (VH), variable heavy chain (VH) domain or heavy chain variable region refer to these light and heavy chain regions, respectively. The terms variable light chain (VL), variable light chain (VL) domain and light chain variable region as referred to herein may be used interchangeably. The terms variable heavy chain (VH), variable heavy chain (VH) domain and heavy chain variable region as referred to herein may be used interchangeably. The Fc (i.e. fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)). The term “antibody” as referred to herein further includes antibody variants such as single domain antibodies. Thus, in embodiments an antibody includes a single monomeric variable antibody domain. Thus, in embodiments, the antibody, includes a variable light chain (VL) domain or a variable heavy chain (VH) domain. In embodiments, the antibody is a variable light chain (VL) domain or a variable heavy chain (VH) domain.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

Techniques for conjugating therapeutic agents to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)). As used herein, the term “antibody-drug conjugate” or “ADC” refers to a therapeutic agent conjugated or otherwise covalently bound to to an antibody.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

The term “multimer” refers to a complex comprising multiple monomers (e.g. a protein complex) associated by noncovalent bonds. The monomers be substantially identical monomers, or the monomers may be different. In embodiments, the multimer is a dimer, a trimer, a tetramer, or a pentamer. Thus, a trimer comprises three monomers associated by noncovalent bonds.

A “ligand” refers to an agent, e.g., a polypeptide or other molecule, capable of binding to a receptor or antibody, antibody variant, antibody region or fragment thereof.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

“Contacting” is used in accordance with its plain ordinary meaning and refers to the process of allowing at least two distinct species (e.g. antibodies and antigens) to become sufficiently proximal to react, interact, or physically touch. It should be appreciated; however, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a pharmaceutical composition as provided herein and a cell. In embodiments contacting includes, for example, allowing a pharmaceutical composition as described herein to interact with a cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

The terms “virus” or “virus particle” are used according to their plain ordinary meaning in the biological arts and refer to a particle including a viral genome (e.g. DNA, RNA, single strand, double strand), a protective coat of proteins (e.g. capsid) and associated proteins, and in the case of enveloped viruses (e.g. herpesvirus), an envelope including lipids and optionally components of host cell membranes, and/or viral proteins. In embodiments, the virus is a rabies virus.

The terms “multiplicity of infection” or “MOI” are used according to its plain ordinary meaning in Virology and refers to the ratio of components to the target (e.g., cell) in a given area. In embodiments, the area is assumed to be homogenous.

“Rabies virus” refers to a member of the group of single stranded, negative sense RNA viruses that are members of the family Rhabdoviridae. The single-stranded RNA genome of Rabies viruses encode the large protein L, phosphoprotein P, matrix protein M, nucleoprotein N, and glycoprotein G. Rabies viruses may infect vertebrate and invertebrate animals, plants, and protzoans, and some infect humans to cause disease. Rabies viruses may acquire ribosomes from their host cells. Rabies virus (RABV) is a member of the family Rhabdoviridae, of which vesicular stomatitis Indiana virus is the prototype. The mammalian disease rabies is caused by lyssaviruses, and embodiments of the present invention directed to “rabies virus glycoproteins” are to be included within the spirit and purview of “lyssavirus glycoproteins”.

The term “replicate” is used in accordance with its plain ordinary meaning and refers to the ability of a cell or virus to produce progeny. A person of ordinary skill in the art will immediately understand that the term replicate when used in connection with DNA, refers to the biological process of producing two identical replicas of DNA from one original DNA molecule. In the context of a virus, the term “replicate” includes the ability of a virus to replicate (duplicate the viral genome and packaging said genome into viral particles) in a host cell and subsequently release progeny viruses from the host cell.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods. Thus, a recombinant protein refers to a protein made by introducing a cell with a nucleic acid that is not typically found in the cell (e.g. non-native DNA). The cells containing the non-native nucleic acid may then transcribe and translate the protein.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The terms “bind” and “bound” as used herein is used in accordance with its plain and ordinary meaning and refers to the association between atoms or molecules. The association can be covalent (e.g., by a covalent bond or linker) or non-covalent (e.g., electrostatic interactions (e.g., ionic bond, hydrogen bond, or halogen bond), van der Waals interactions (e.g., dipole-dipole, dipole-induced dipole, or London dispersion), ring stacking (pi effects), hydrophobic interactions, and the like).

As used herein, the term “conjugated” when referring to two moieties means the two moieties are bonded, wherein the bond or bonds connecting the two moieties may be covalent or non-covalent. In embodiments, the two moieties are covalently bonded to each other (e.g., directly or through a covalently bonded intermediary). In embodiments, the two moieties are non-covalently bonded (e.g., through ionic bond(s), van der Waals bond(s)/interactions, hydrogen bond(s), polar bond(s), or combinations or mixtures thereof).

As used herein, the terms “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH2, —C(O)OH, —N-hydroxysuccinimide, or-maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g. phosphines) to form, for example, phosphate diester bonds.
(n) azides coupled to alkynes using copper catalyzed cycloaddition click chemistry.
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

As defined herein, the term “inhibition”, “inhibit”, “inhibiting” and the like in reference to cell proliferation (e.g., cancer cell proliferation) means negatively affecting (e.g., decreasing proliferation) or killing the cell. In embodiments, inhibition refers to reduction of a disease or symptoms of disease (e.g., cancer, cancer cell proliferation). Thus, inhibition includes, at least in part, partially or totally blocking stimulation, decreasing, preventing, or delaying activation, or inactivating, desensitizing, or down-regulating signal transduction or enzymatic activity or the amount of a protein. Similarly an “inhibitor” is a compound or protein that inhibits a receptor or another protein, e.g., by binding, partially or totally blocking, decreasing, preventing, delaying, inactivating, desensitizing, or down-regulating activity (e.g., a receptor activity or a protein activity).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, synoviocytes, synovial fluid, synovial tissue, fibroblast-like synoviocytes, macrophagelike synoviocytes, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with the compounds or methods provided herein.

The term “associated” or “associated with” in the context of a substance or substance activity or function associated with a disease (e.g., rabies virus infection) means that the disease is caused by (in whole or in part), or a symptom of the disease is caused by (in whole or in part) the substance or substance activity or function. Alternatively, the substance may be an indicator of the disease. Thus, an associated substance may serve as a means of targeting disease tissue.

A “therapeutic agent” as referred to herein, is a composition useful in treating or preventing a disease such as a viral infection (e.g. rabies virus). In embodiments, the therapeutic agent is an anti-viral agent. “Anti-viral agent” is used in accordance with its plain ordinary meaning and refers to a composition (e.g. compound, drug, antagonist, inhibitor, modulator) having anti-viral properties or the ability to inhibit viral infection. In embodiments, an anti-viral agent targets a viral protein. In embodiments, an anti-viral agent inhibits viral entry into a host cell. In embodiments, an anti-viral agent inhibits replication of viral components. In embodiments, an anti-viral inhibits release of viral particles. In embodiments, an anti-viral inhibits assembly of viral particles.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The term “prevent” refers to a decrease in the occurrence of a disease or disease symptoms in a patient. As indicated above, the prevention may be complete (no detectable symptoms) or partial, such that fewer symptoms are observed than would likely occur absent treatment.

As used herein, a “symptom” of a disease includes any clinical or laboratory manifestation associated with the disease, and is not limited to what a subject can feel or observe.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from binding assays or cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols. The preparations may also be combined with inhaled mucolytics (e.g., rhDNase, as known in the art) or with inhaled bronchodilators (short or long acting beta agonists, short or long acting anticholinergics), inhaled corticosteroids, or inhaled antibiotics to improve the efficacy of these drugs by providing additive or synergistic effects. The compositions of the present invention can be delivered transdermally, by a topical route, formulated as applicator sticks, solutions, suspensions, emulsions, gels, creams, ointments, nanoparticles, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J Hosp. Pharm. 46:1576-1587, 1989). The compositions of the present invention can also be delivered as nanoparticles.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The term “vaccine” refers to a composition that can provide active acquired immunity to and/or therapeutic effect (e.g. treatment) of a particular disease or a pathogen. A vaccine typically contains one or more agents that can induce an immune response in a subject against a pathogen or disease, i.e. a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g. preventing or ameliorating the effects of a future infection by any natural or pathogen, or of an anticipated occurrence of cancer in a predisposed subject) or therapeutic (e.g., treating cancer in a subject who has been diagnosed with the cancer). The administration of vaccines is referred to vaccination. In embodiments, a vaccine composition can provide nucleic acid, e.g. mRNA that encodes antigenic molecules (e.g. peptides) to a subject. The nucleic acid that is delivered via the vaccine composition in the subject can be expressed into antigenic molecules and allow the subject to acquire immunity against the antigenic molecules. In the context of the vaccination against infectious disease, the vaccine composition can provide mRNA encoding antigenic molecules that are associated with a certain pathogen, e.g. one or more peptides that are known to be expressed in the pathogen (e.g. pathogenic bacterium or virus). In the context of cancer vaccine, the vaccine composition can provide mRNA encoding certain peptides that are associated with cancer, e.g. peptides that are substantially exclusively or highly expressed in cancer cells as compared to normal cells. The subject, after vaccination with the cancer vaccine composition, can have immunity against the peptides that are associated with cancer and kill the cancer cells with specificity.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

The term “adjuvant” refers to a compound that when administered in conjunction with the agents provided herein including embodiments thereof, augments the agent's immune response. Adjuvants can augment an immune response by several mechanisms including lymphocyte recruitment, stimulation of B and/or T cells, and stimulation of macrophages. The adjuvant increases the titer of induced antibodies and/or the binding affinity of induced antibodies relative to the situation if the immunogen were used alone. A variety of adjuvants can be used in combination with the agents provided herein including embodiments thereof, to elicit an immune response. Preferred adjuvants augment the intrinsic response to an immunogen without causing conformational changes in the immunogen that affect the qualitative form of the response. Preferred adjuvants include aluminum hydroxide and aluminum phosphate, 3 De-O-acylated monophosphoryl lipid A (MPL™) (see GB 2220211 (RIBI ImmunoChem Research Inc., Hamilton, Montana, now part of Corixa). Stimulon™ QS-21 is a triterpene glycoside or saponin isolated from the bark of the Quillaja Saponaria Molina tree found in South America (see Kensil et al., in Vaccine Design: The Subunit and Adjuvant Approach (eds. Powell & Newman, Plenum Press, NY, 1995); U.S. Pat. No. 5,057,540), (Aquila BioPharmaceuticals, Framingham, MA). Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Another adjuvant is CpG (WO 98/40100). Adjuvants can be administered as a component of a therapeutic composition with an active agent or can be administered separately, before, concurrently with, or after administration of the therapeutic agent.

Other adjuvants contemplated for the invention are saponin adjuvants, such as Stimulon™ (QS-21, Aquila, Framingham, MA) or particles generated therefrom such as ISCOMs (immunostimulating complexes) and ISCOMATRIX. Other adjuvants include RC-529, GM-CSF and Complete Freund's Adjuvant (CFA) and Incomplete Freund's Adjuvant (IFA). Other adjuvants include cytokines, such as interleukins (e.g., IL-1 α and β peptides, IL-2, IL-4, IL-6, IL-12, IL-13, and IL-15), macrophage colony stimulating factor (M-CSF), granulocyte-macrophage colony stimulating factor (GM-CSF), tumor necrosis factor (TNF), chemokines, such as MIP1α and β and RANTES. Another class of adjuvants is glycolipid analogues including N-glycosylamides, N-glycosylureas and N-glycosylcarbamates, each of which is substituted in the sugar residue by an amino acid, as immuno-modulators or adjuvants (see U.S. Pat. No. 4,855,283). Heat shock proteins, e.g., HSP70 and HSP90, may also be used as adjuvants.

The term “immune response” used herein encompasses, but is not limited to, an “adaptive immune response”, also known as an “acquired immune response” in which adaptive immunity elicits immunological memory after an initial response to a specific pathogen or a specific type of cells that is targeted by the immune response, and leads to an enhanced response to that target on subsequent encounters. The induction of immunological memory can provide the basis of vaccination.

The term “immunogenic” or “antigenic” refers to a compound or composition that induces an immune response, e.g., cytotoxic T lymphocyte (CTL) response, a B cell response (for example, production of antibodies that specifically bind the epitope), an NK cell response or any combinations thereof, when administered to an immunocompetent subject. Thus, an immunogenic or antigenic composition is a composition capable of eliciting an immune response in an immunocompetent subject. For example, an immunogenic or antigenic composition can include one or more immunogenic epitopes associated with a pathogen or a specific type of cells that is targeted by the immune response. In addition, an immunogenic composition can include isolated nucleic acid constructs (such as DNA or RNA) that encode one or more immunogenic epitopes of the antigenic polypeptide that can be used to express the epitope(s) (and thus be used to elicit an immune response against this polypeptide or a related polypeptide associated with the targeted pathogen or type of cells).

### Rabies Virus Glycoprotein Compositions

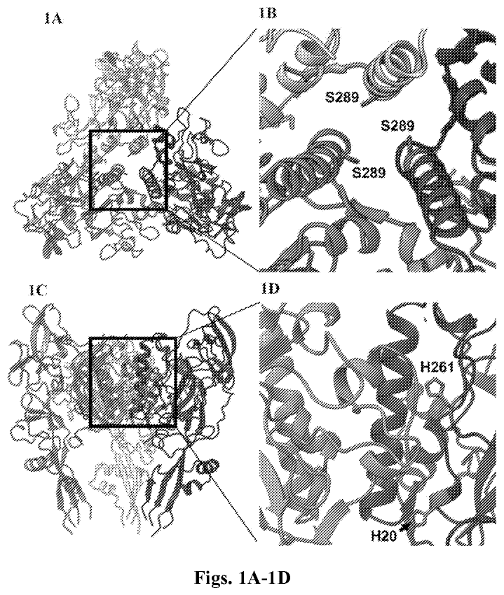

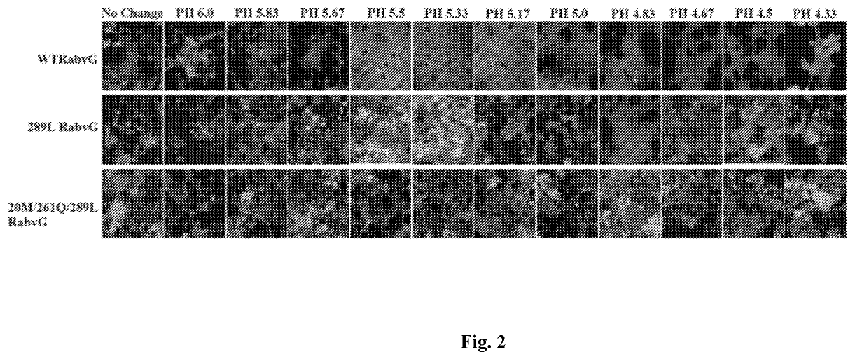

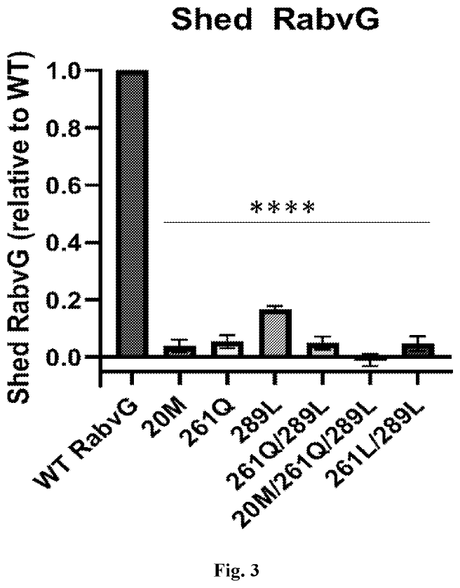

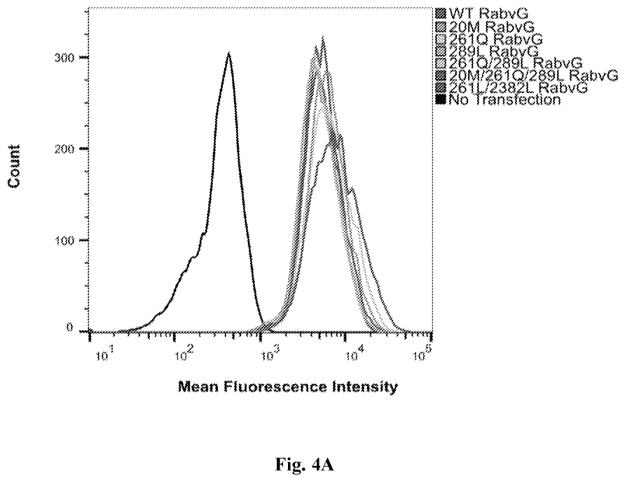

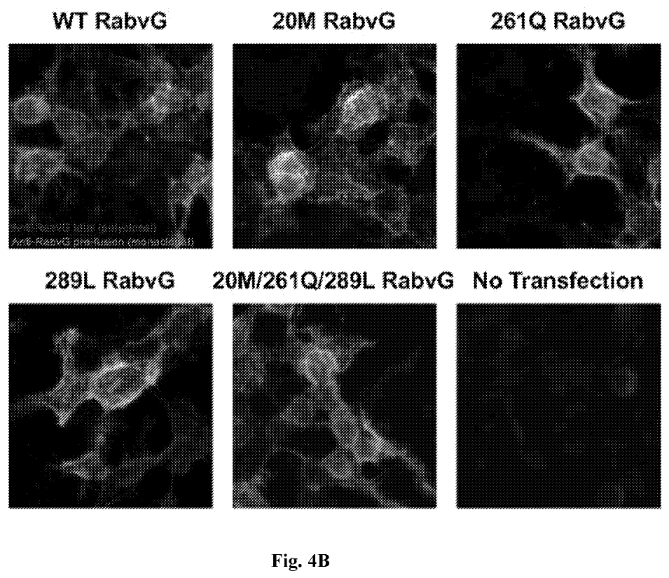

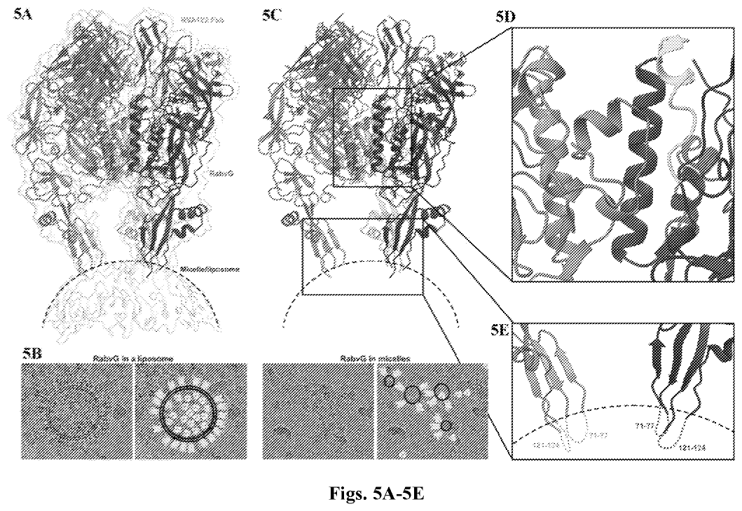

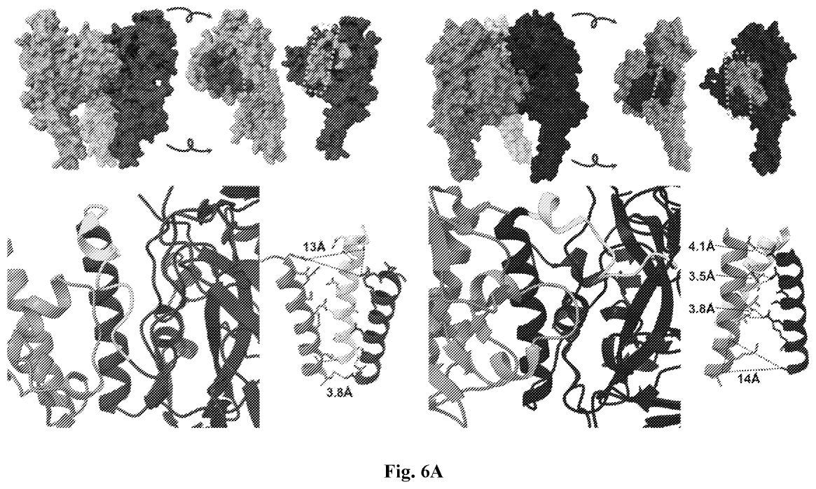

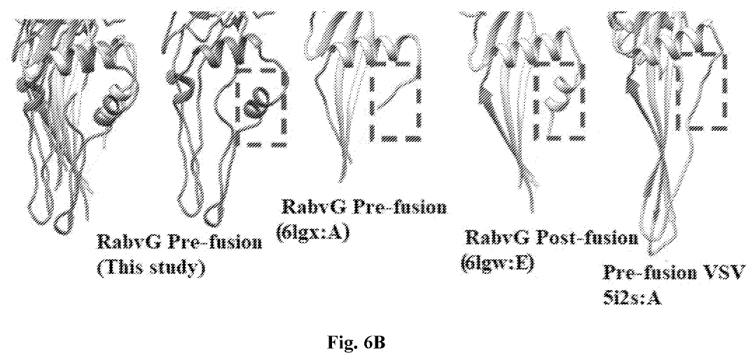

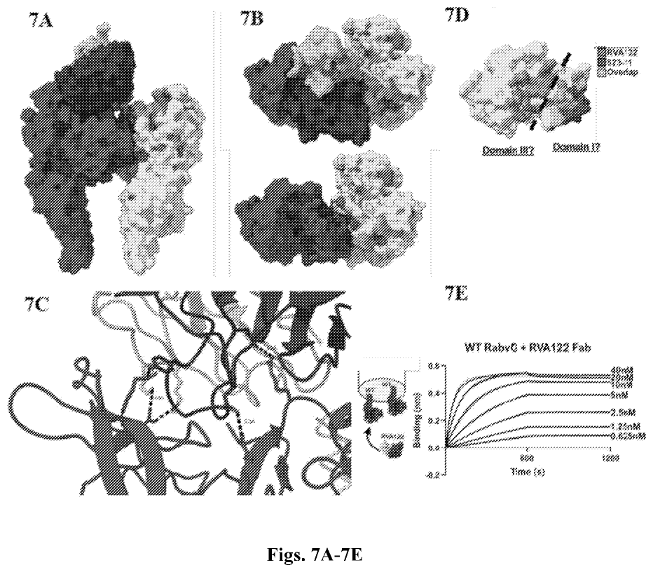

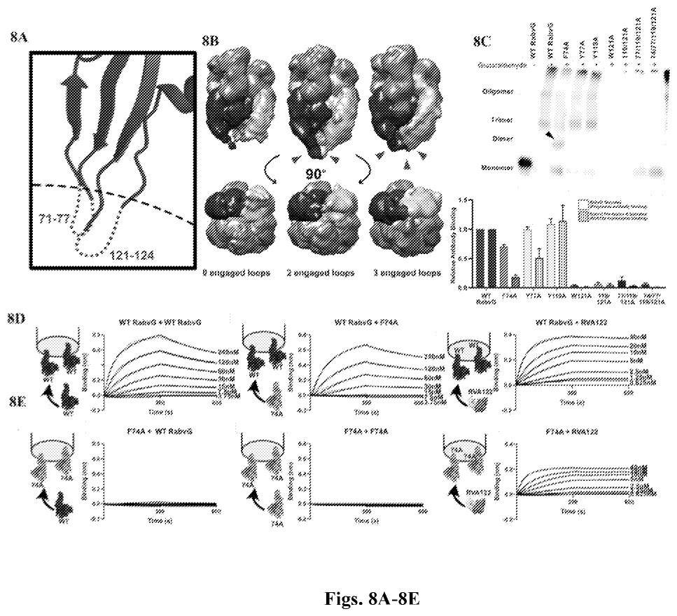

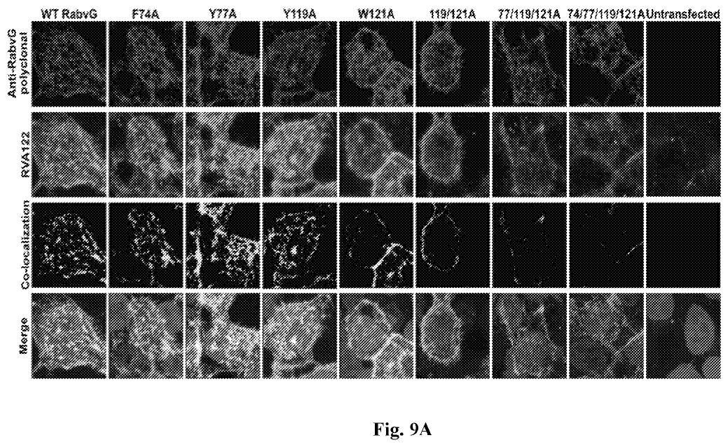

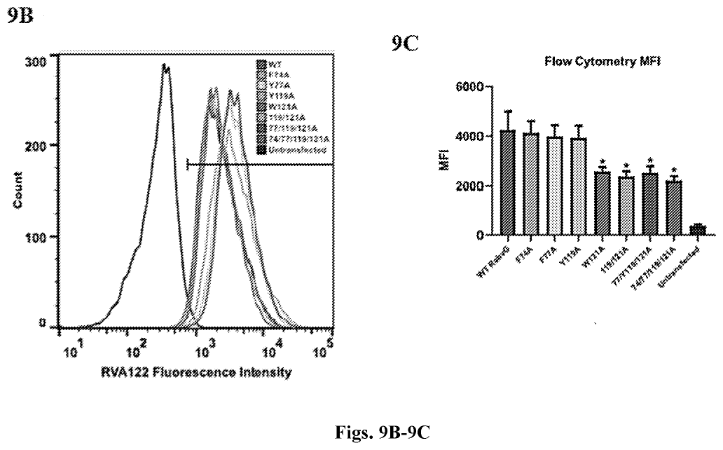

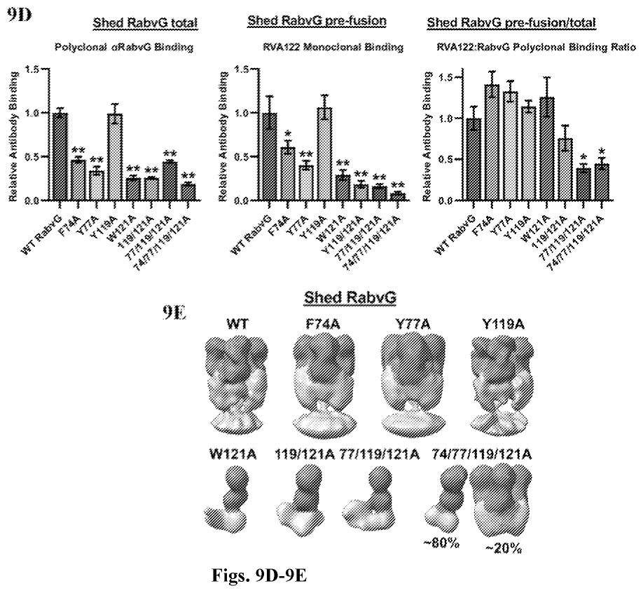

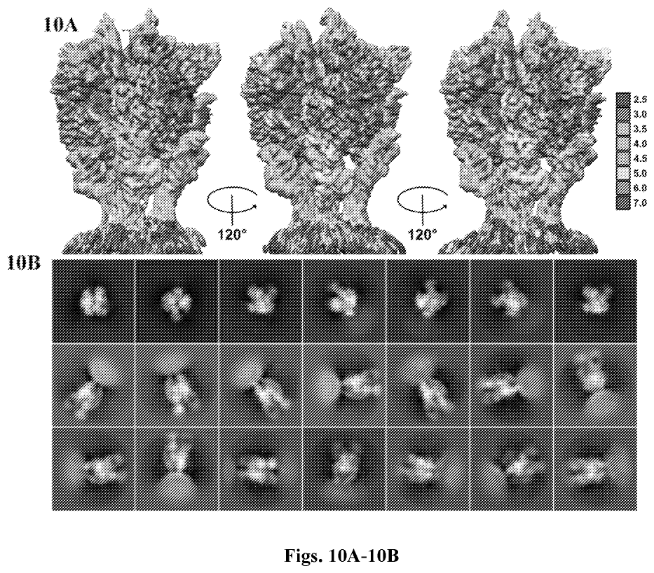

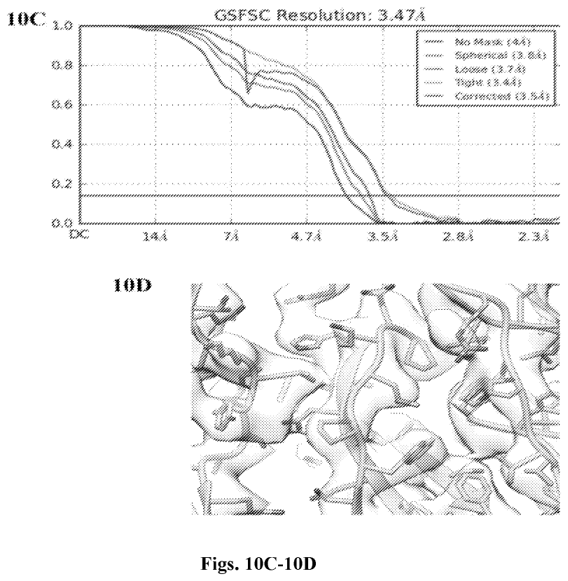

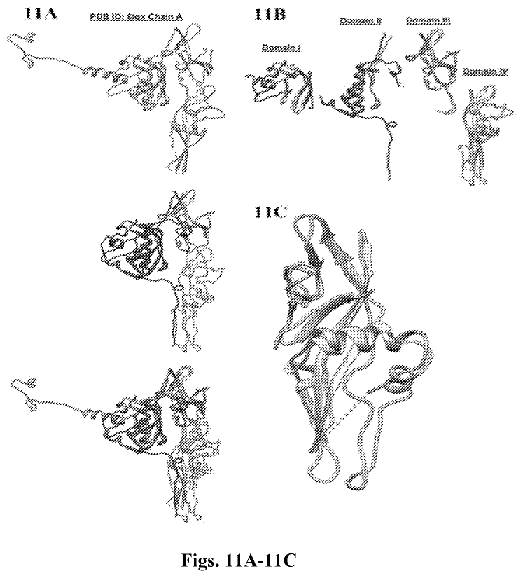

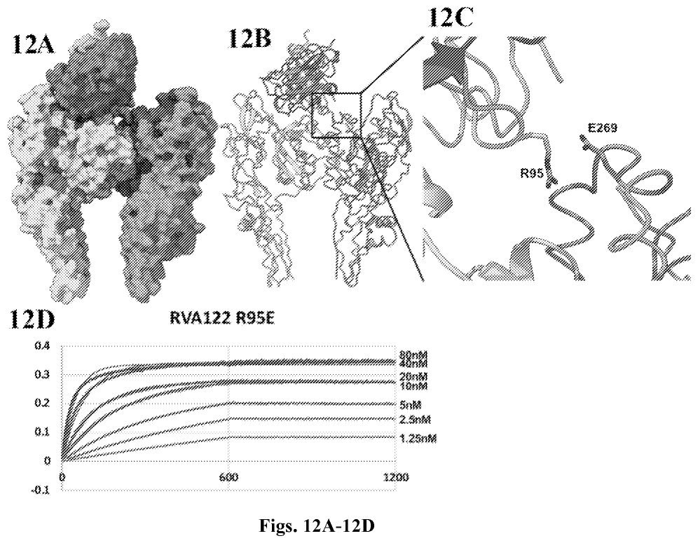

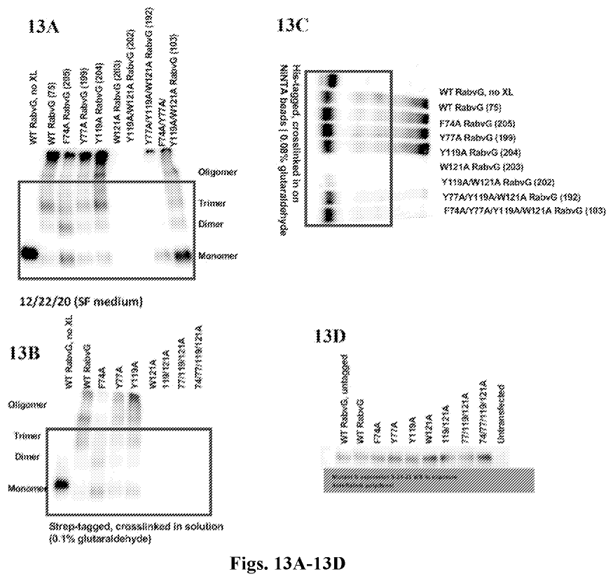

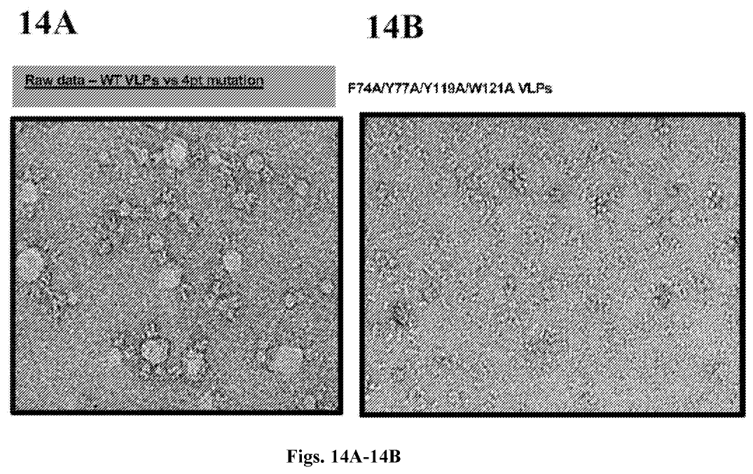

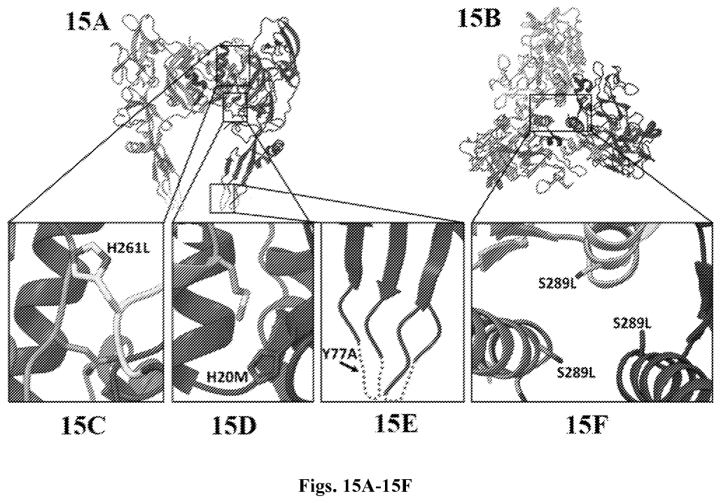

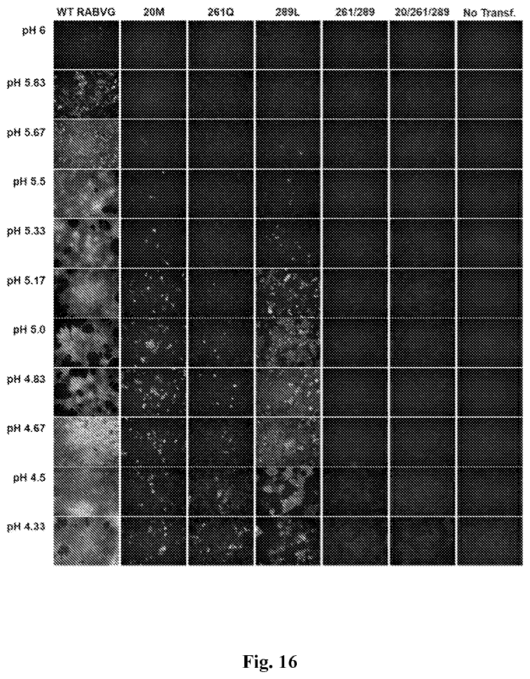

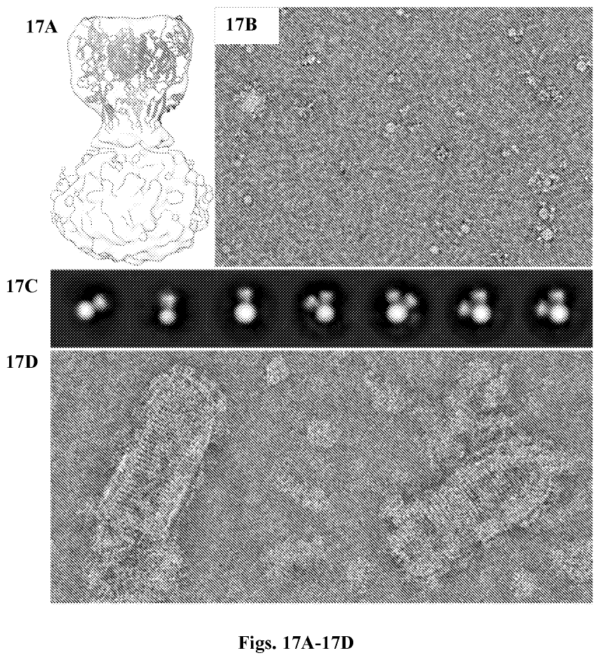

The recombinant rabies virus glycoprotein provided herein including embodiments thereof include a rabies virus glycoprotein and, optionally, a trimerization domain. The inventors have determined the structure of the trimeric rabies virus glycoprotein and introduced a series of point mutations to the protein in order to stabilize it in its trimeric, pre-fusion conformation. The mutations consist of changes to four residues on the trimeric interface of RabvG: His20Met, His261Gln, Tyr77Ala, and Ser289Leu (FIG. 1). These mutations increase the proportion of soluble RabvG in the pre-fusion conformation by ˜700% compared to the wild-type glycoprotein (Table 1) and inhibit the transition from pre-fusion to post-fusion in the full-length glycoprotein (FIG. 2), indicating increased protein stability. 20Met/261Gln/289Leu mutation reduces secretion of the soluble RabvG ectodomain into supernatant (Table 1) and shedding/cleavage of full-length RabvG from cells (FIG. 3), but does not affect the expression of full-length RabvG on the surface of transfected cells (FIG. 4), making it an ideal candidate for an RNA vaccine encoding full-length glycoprotein. Further, 77Ala aids in the purifying of stabilize glycoprotein ectodomain, resulting in a formulation better suited for structural biology or, as embodiments of the present disclosure set forth, a protein subunit vaccine.

As used herein, the term “trimerization domain” refers to a protein or peptide domain that is capable of non-covalently binding two other trimerization domains to form a protein trimer. As used herein, trimerization domain refers to a protein that is not naturally encoded by the rabies virus genome (e.g. an exogenous trimerization domain). Thus, in an aspect is provided a recombinant rabies virus glycoprotein including a rabies virus glycoprotein ectodomain and a trimerization domain.

In embodiments, the recombinant rabies virus glycoprotein includes the amino acid sequence of SEQ ID NO:1 or SEQ ID NO:2. In embodiments, the recombinant rabies virus glycoprotein is the amino acid sequence of SEQ ID NO:1 or SEQ ID NO:2. In embodiments, the recombinant rabies virus glycoprotein is a polypeptide having at least 85%, 86%, 87%, 88%, 89%, 90%, 91%, 92%, 93%, 94%, 95%, 96%, 97%, 98%, 99%, or 100% sequence identity across the whole sequence or a portion of the sequence of SEQ ID NO: 1. In embodiments, the recombinant rabies virus glycoprotein is a polypeptide having 85-86%, 86-87%, 87-88%, 88-89%, 89-90%, 90-91%, 91-92%, 92-93%, 93-94%, 94-95%, 95-96%, 96-97%, 97-98%, 98-99%, or 99-100% sequence identity across the whole sequence or a portion of the sequence of SEQ ID NO:1 or SEQ ID NO:2.

In embodiments, the recombinant rabies virus glycoprotein includes the amino acid sequence of SEQ ID NO:2. In embodiments, the recombinant rabies virus glycoprotein is the amino acid sequence of SEQ ID NO:2. In embodiments, the recombinant rabies virus glycoprotein is a polypeptide having at least 85%, 86%, 87%, 88%, 89%, 90%, 91%, 92%, 93%, 94%, 95%, 96%, 97%, 98%, 99%, or 100% sequence identity across the whole sequence or a portion of the sequence of SEQ ID NO:2. In embodiments, the recombinant rabies virus glycoprotein is a polypeptide having 85-86%, 86-87%, 87-88%, 88-89%, 89-90%, 90-91%, 91-92%, 92-93%, 93-94%, 94-95%, 95-96%, 96-97%, 97-98%, 98-99%, or 99-100% sequence identity across the whole sequence or a portion of the sequence of SEQ ID NO:2.

The recombinant rabies virus glycoprotein provided herein including embodiments thereof is capable of forming a glycoprotein trimer. Applicant has discovered that neutralizing antibodies may recognize and bind the glycoprotein trimer. Thus, in an aspect is provided a recombinant rabies virus glycoprotein trimer. In further embodiments, the trimer includes three of the recombinant rabies virus glycoproteins provided herein including embodiments thereof, wherein the three recombinant rabies virus glycoproteins are optionally bound by non-covalent attachment of the trimerization domains.

### Nucleic Acid Compositions

In an aspect is provided a nucleic acid encoding the recombinant rabies virus glycoprotein provided herein including embodiments thereof. The nucleic acid provided herein, including embodiments thereof, may be loaded into an expression vector such that the nucleic acid may be delivered to cells. Thus, in an aspect, an expression vector including the nucleic acid provided herein, including embodiments thereof, is provided. It is contemplated that the nucleic acid may be loaded into any expression vector useful for delivering the nucleic acid to cells either in vivo or in vitro.

As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically. Replication-incompetent viral vectors or replication-defective viral vectors refer to viral vectors that are capable of infecting their target cells and delivering their viral payload, but then fail to continue the typical lytic pathway that leads to cell lysis and death.

### Cells

In an aspect is provided a cell including the recombinant rabies virus glycoprotein provided herein including embodiments thereof or the glycoprotein trimer provided herein including embodiments thereof. In another aspect is provided a cell including the nucleic acid provided herein including embodiments thereof. In embodiments, the cell is a human cell.

### Vaccine Compositions

The recombinant rabies virus glycoprotein and glycoprotein trimer provided herein including embodiments thereof are contemplated to be particularly effective in vaccine compositions for treating and/or preventing rabies virus infections. Applicant has found that the glycoprotein trimer is recognized by anti-glycoprotein antibodies, and may be a target for antibody-mediated neutralization of rabies virus infection. Thus, in an aspect is provided a vaccine composition including the recombinant rabies virus glycoprotein provided herein including embodiments thereof and a pharmaceutically acceptable excipient. In another aspect is provided a vaccine composition including the glycoprotein trimer provided herein including embodiments thereof and a pharmaceutically acceptable excipient.

In embodiments, the vaccine composition further includes one or more of a stabilizer, an adjuvant, and a preservative. In embodiments, the vaccine composition includes a stabilizer. In embodiments, the vaccine composition includes a preservative. In embodiments, the vaccine further comprises an adjuvant. In embodiments, the adjuvant is a gel-type, microbial, particulate, oil-emulsion, surfactant-based, or synthetic adjuvant. In embodiments, the adjuvant is aluminum hydroxide/phosphate, calcium phosphate, muramyl dipeptide (MDP), a bacterial exotoxin, an endotoxin-based adjuvant, a biodegradable adjuvant, polymer microspheres, immunostimulatory complexes (ISCOMs), liposomes, Freund's incomplete adjuvant, microfluidized emulsions, saponins, muramyl peptide derivatives, nonionic block copolymers, polyphosphazene (PCPP), synthetic polynucleotide, or a thalidomide derivative. In embodiments, the adjuvant is a CpG oligonucleotide. In embodiments, the adjuvant comprises a BCG sequence. In embodiments, the adjuvant is a tetanus toxoid.

### Methods

The recombinant rabies virus glycoprotein provided herein including embodiments thereof is particularly useful for treating or preventing rabies virus infections. Thus, in an aspect is provided a method of treating or preventing a viral disease in a subject in need of such treatment or prevention, the method including administering a therapeutically or prophylactically effective amount of the recombinant rabies virus glycoprotein provided herein including embodiments thereof to the subject. In another aspect is provided a method of treating or preventing a viral disease in a subject in need of such treatment or prevention, the method including administering a therapeutically or prophylactically effective amount of the glycoprotein trimer provided herein including embodiments thereof to the subject.

In an aspect is provided a method for immunizing a subject susceptible to a viral disease, the method including administering the recombinant rabies virus glycoprotein provided herein including embodiments thereof to a subject under conditions such that antibodies directed to the rabies virus glycoprotein or a fragment thereof are produced. In an aspect is provided a method for immunizing a subject susceptible to a viral disease, the method including administering the glycoprotein trimer provided herein including embodiments thereof to a subject under conditions such that antibodies directed to the glycoprotein trimer or a fragment thereof are produced.

The recombinant rabies virus glycoproteins and glycoprotein trimers provided herein including embodiments thereof are contemplated to be useful for as research tools in a variety of clinical or research applications. These include, e.g., characterizing the desired types of antibodies in a discovery effort for immunotherapeutics or diagnostics, and characterizing the desired types of antibody responses elicited by a vaccine or a natural infection. Thus, in an aspect is provided a method of diagnosing rabies virus infection in a subject, the method including: (a) contacting a biological sample obtained from the subject with the recombinant rabies virus glycoprotein provided herein including embodiments thereof, and (b) detecting binding of one or more antibodies to the recombinant rabies virus glycoprotein, thereby diagnosing rabies virus infection in the subject. In another aspect is provided a method of diagnosing rabies virus infection in a subject, the method including: (a) contacting a biological sample obtained from the subject with the glycoprotein trimer provided herein including embodiments thereof, and (b) detecting binding of one or more antibodies to the glycoprotein trimer, thereby diagnosing rabies virus infection in the subject.

In embodiments, the recombinant rabies virus glycoproteins and glycoprotein trimers are used to diagnose rhabdoviridae infections (e.g., RABV infections), including lyssavirus infections. In embodiments, a biological sample is typically obtained from subjects suspected of having a rhabdoviral, lyssaviral, or rabies virus infection. The biological sample may be any tissue or liquid sample from the subject. In embodiments, the sample is a blood sample, e.g., whole blood, plasma or serum. The sample is then contacted with a recombinant rabies virus glycoprotein or glycoprotein trimer provided herein including embodiments thereof to allow detection of both neutralizing and non-neutralizing antibodies against a rabies virus GP. In embodiments, the recombinant rabies virus glycoproteins and glycoprotein trimers provided herein including embodiments thereof can be employed to evaluate effectiveness of rabies virus vaccines. In these applications, a subject who has been immunized with a test vaccine against a rabies virus (e.g., RABV) is examined for production of antibodies against the virus, esp. neutralizing antibodies. For example, a blood sample can be taken from the subject, which can then be contacted with a recombinant rabies virus glycoproteins or glycoprotein trimer. Rabies virus specific antibodies that react with the immunogen (e.g. recombinant rabies virus glycoproteins, glycoprotein trimers) can then be assessed qualitatively and quantitatively. For example, the antigen-antibody immune complexes can be readily isolated from the blood sample. The types of the antibodies present in the blood sample can be analyzed qualitatively by comparing them to antibodies known in the art, e.g., all known RABV neutralizing antibodies. These can be accomplished by employing the various techniques that have been routinely practiced in the art or exemplified herein. Methods for analyzing and identifying antibodies are described in greater detail in Robinson et al., Nat. Comm. 7:11544, 2016; Hastie et al., J. Virol. 90:4556-62, 2016; Hastie et al., Science 356:923-928, 2017; Li et al., Vaccine. 355172-5178, 2017; Sommerstein et al., PLoS Pathog. 11:e1005276, 2015; Bukbuk et al., Trans. R. Soc. Trop. Med. Hyg. 108:768-73, 2014; and Shaffer et al., PLoS Negl. Trop. Dis. 8:e2748, 2014; which are incorporated herein by reference in their entirety for all purposes. The recombinant rabies virus glycoproteins and glycoprotein trimers provided herein including embodiments thereof are contemplated to be useful for methods of quantitatively examining neutralizing antibodies produced in the subject as a result of vaccination. Such an analysis can also be readily performed with standard immunological protocols well known in the art (e.g., ELISA). By detection of virtually all neutralizing antibodies and also non-neutralizing antibodies, the engineered rabies viral immunogens of the invention thus enable both qualitative and quantitative evaluation of antibody responses elicited by a vaccine in a subject or a group of subjects.

The recombinant rabies virus glycoproteins and glycoprotein trimers provided herein including embodiments thereof can be used in evaluating the effectiveness of a rabies virus vaccine in a subject. In certain embodiments, this includes contacting a biological sample from a subject who has been administered with a vaccine for a rabies virus with the recombinant rabies virus glycoproteins and glycoprotein trimers provided herein, (b) detecting antibodies in the biological sample that specifically bind to the recombinant rabies virus glycoproteins and glycoprotein trimers provided, and (c) performing quantitative and qualitative analysis of the antibodies detected in the biological sample, thereby evaluating effectiveness of the rabies virus vaccine in the subject. The recombinant rabies virus glycoproteins and glycoprotein trimers provided herein including embodiments thereof can be provided as components of diagnostic kits.

In embodiments, the kit includes components including packaging and reagents. In embodiments, the reagents include buffers, substrates, antibodies or ligands (e.g. control antibodies or ligands), and detection reagents. In embodiments, the kit includes an instruction sheet.

### Veterinary Use

The present application provides pharmaceutical compositions administered to a veterinary subject comprising a recombinant rabies virus glycoprotein. In embodiments, the pharmaceutical composition is a vaccine. In embodiments, the veterinary subject is a mammal. In further embodiments, the mammal is a canine, cat (e.g., Felis catus), ferret, rabbit, cattle, horse, monkey, coyote, or any mammal susceptible to being infected with Rabies lyssavirus.

Vaccines contemplated herein, may be administered to a veterinary subject orally, parenterally, subcutaneously, intramuscularly, intranasally, or topically. In embodiments, delivery of the vaccine relies on the presence of dendritic cells in the tissues that take up the antigen (antigen-presenting cell) via endocytosis or phagocytosis and present the foreign antigen to inducer or helper T cells to elicit an immune response.

### P Embodiments

P embodiment 1: A recombinant rabies virus glycoprotein comprising a rabies virus glycoprotein comprising one or more of the following mutations: His20Met, His261Gln, Tyr77Ala, and Ser289Leu.

P embodiment 2: A recombinant rabies virus glycoprotein comprising a rabies virus glycoprotein having an amino acid sequence of SEQ ID NO:1 or SEQ ID NO:2.

P embodiment 3: A recombinant rabies virus glycoprotein comprising a rabies virus glycoprotein having an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:1 or SEQ ID NO:2.

P embodiment 4: A glycoprotein trimer comprising the recombinant rabies virus glycoprotein of any one of embodiments 1-3.

P embodiment 5: A nucleic acid encoding the recombinant rabies virus glycoprotein of any one of embodiments 1-3, or the glycoprotein trimer of embodiment 4.

P embodiment 6: The nucleic acid of embodiment 4, further comprising a vector.

P embodiment 7: A cell comprising the recombinant rabies virus glycoprotein of any one of embodiments 1-3 or the glycoprotein trimer of embodiment 4.

P embodiment 8: A cell comprising the nucleic acid of embodiment 4.

P embodiment 9: The cell of embodiment 13, wherein the cell is a human cell.

P embodiment 10: A vaccine composition comprising the recombinant rabies virus glycoprotein of any one of embodiments 1-3 and a pharmaceutically acceptable excipient.

P embodiment 11: A vaccine composition comprising the glycoprotein trimer of embodiment 4 and a pharmaceutically acceptable excipient.

P embodiment 12: The vaccine composition of embodiment 9 or 10, further comprising an adjuvant.

P embodiment 13: A method of treating or preventing a viral disease in a subject in need of such treatment or prevention, said method comprising administering a therapeutically or prophylactically effective amount of the recombinant rabies virus glycoprotein of any one of embodiments 1-3 to the subject.

P embodiment 14: A method of treating or preventing a viral disease in a subject in need of such treatment or prevention, said method comprising administering a therapeutically or prophylactically effective amount of the glycoprotein trimer of embodiment 4 to the subject.

P embodiment 15: The method of embodiment 14, wherein said viral disease is caused by a member of the Rhabdoviridae family.

P embodiment 16: The method of embodiment 14, wherein said viral disease is a lyssavirus.

P embodiment 17: The method of embodiment 14 or embodiment 15, wherein said viral disease is a rabies virus.

P embodiment 18: A method for immunizing a subject susceptible to a viral disease, comprising administering the recombinant rabies virus glycoprotein of any one of embodiments 1-3 to a subject under conditions such that antibodies directed to said rabies virus glycoprotein or a fragment thereof are produced.

P embodiment 19: A method for immunizing a subject susceptible to a viral disease, comprising administering the glycoprotein trimer of embodiment 4 to a subject under conditions such that antibodies directed to said glycoprotein trimer or a fragment thereof are produced.

P embodiment 20: A method of diagnosing rabies virus infection in a subject, the method comprising: (a) contacting a biological sample obtained from the subject with the recombinant rabies virus glycoprotein of any one of embodiments 1-3, and (b) detecting binding of one or more antibodies to said recombinant rabies virus glycoprotein, thereby diagnosing rabies virus infection in said subject.

P embodiment 21: A method of diagnosing rabies virus infection in a subject, the method comprising: (a) contacting a biological sample obtained from the subject with the glycoprotein trimer of embodiment 4, and (b) detecting binding of one or more antibodies to said glycoprotein trimer, thereby diagnosing rabies virus infection in said subject.

### Embodiments

Embodiment 1: A recombinant rabies virus glycoprotein comprising a rabies virus glycoprotein comprising one or more of the following mutations: His20Met, His261Gln, Tyr77Ala, and Ser289Leu.

Embodiment 2: The recombinant rabies virus glycoprotein of embodiment 1, wherein the mutation is at least His20Met.

Embodiment 3: The recombinant rabies virus glycoprotein of embodiment 1, wherein the mutation is at least His261Gln.

Embodiment 4: The recombinant rabies virus glycoprotein of embodiment 1, wherein the mutation is at least Tyr77Ala.

Embodiment 5: The recombinant rabies virus glycoprotein of embodiment 1, wherein the mutation is at least Ser289Leu.

Embodiment 6: A recombinant rabies virus glycoprotein comprising a rabies virus glycoprotein having an amino acid sequence of SEQ ID NO:1 or SEQ ID NO:2.

Embodiment 7: The recombinant rabies virus glycoprotein of embodiment 6, wherein the rabies virus glycoprotein comprises an amino acid sequence as set forth in SEQ ID NO:1.

Embodiment 8: The recombinant rabies virus glycoprotein of embodiment 6, wherein the rabies virus glycoprotein comprises an amino acid sequence as set forth in SEQ ID NO:2.

Embodiment 9: A recombinant rabies virus glycoprotein comprising a rabies virus glycoprotein having an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:1 or SEQ ID NO:2.

Embodiment 10: A glycoprotein trimer comprising the recombinant rabies virus glycoprotein of any one of embodiments 1-9.

Embodiment 11: A nucleic acid encoding the recombinant rabies virus glycoprotein of any one of embodiments 1-9 or the glycoprotein trimer of embodiment 10

Embodiment 12: The nucleic acid of embodiment 11 further comprising a vector.

Embodiment 13: A cell comprising the recombinant rabies virus glycoprotein of any one of embodiments 1-9 or the glycoprotein trimer of embodiment 10.

Embodiment 14: A cell comprising the nucleic acid of embodiment 11.

Embodiment 15: The cell of embodiment 13, wherein the cell is a human cell.

Embodiment 16: A vaccine composition comprising the recombinant rabies virus glycoprotein of any one of embodiments 1-9 and a pharmaceutically acceptable excipient.

Embodiment 17: A vaccine composition comprising the glycoprotein trimer of embodiment 10 and a pharmaceutically acceptable excipient.

Embodiment 18: The vaccine composition of embodiment 16 or 17 further comprising an adjuvant.

Embodiment 19: A method of treating or preventing a viral disease in a subject in need of such treatment or prevention, said method comprising administering a therapeutically or prophylactically effective amount of the recombinant rabies virus glycoprotein of any one of embodiments 1-9 to the subject.

Embodiment 20: A method of treating or preventing a viral disease in a subject in need of such treatment or prevention, said method comprising administering a therapeutically or prophylactically effective amount of the glycoprotein trimer of embodiment 10 to the subject.

Embodiment 21: The method of embodiment 19, wherein said viral disease is caused by a member of the Rhabdoviridae family.

Embodiment 22: The method of embodiment 21, wherein said viral disease is a lyssavirus.

Embodiment 23: The method of embodiment 21 or embodiment 22, wherein said viral disease is a rabies virus.

Embodiment 24: The method of embodiment 19, wherein the subject is a mammal.

Embodiment 25: The method of embodiment 24, wherein the mammal is human.

Embodiment 26: The method of embodiment 24, wherein the mammal is selected from a canine, cat, ferret, rabbit, cattle, horse, monkey, and coyote.

Embodiment 27: A method for immunizing a subject susceptible to a viral disease, comprising administering the recombinant rabies virus glycoprotein of any one of embodiments 1-9 to a subject under conditions such that antibodies directed to said rabies virus glycoprotein or a fragment thereof are produced.

Embodiment 28: The method of embodiment 27, wherein the subject is a mammal.

Embodiment 29: The method of embodiment 28 wherein the mammal is a human.

Embodiment 30: The method of embodiment 28, wherein the mammal is selected from a canine, cat, ferret, rabbit, cattle, horse, monkey, and coyote.

Embodiment 31: A method for immunizing a subject susceptible to a viral disease, comprising administering the glycoprotein trimer of embodiment 10 to a subject under conditions such that antibodies directed to said glycoprotein trimer or a fragment thereof are produced.

Embodiment 32: The method of embodiment 31, wherein the subject is a mammal.

Embodiment 33: The method of embodiment 32, wherein the mammal is a human.

Embodiment 34: The method of embodiment 32, wherein the mammal is selected from a canine, cat, ferret, rabbit, cattle, horse, monkey, and coyote.

Embodiment 35: A method of diagnosing rabies virus infection in a subject, the method comprising: (a) contacting a biological sample obtained from the subject with the recombinant rabies virus glycoprotein of any one of embodiments 1-9, and (b) detecting binding of one or more antibodies to said recombinant rabies virus glycoprotein, thereby diagnosing rabies virus infection in said subject

Embodiment 36: The method of embodiment 35, wherein the subject is a mammal.

Embodiment 37: The method of embodiment 36, wherein the mammal is a human.

Embodiment 38: The method of embodiment 36, wherein the mammal is selected from a canine, cat, ferret, rabbit, cattle, horse, monkey, and coyote.

Embodiment 39: A method of diagnosing rabies virus infection in a subject, the method comprising: (a) contacting a biological sample obtained from the subject with the glycoprotein trimer of embodiment 10, and (b) detecting binding of one or more antibodies to said glycoprotein trimer, thereby diagnosing rabies virus infection in said subject.

Embodiment 40: The method of embodiment 39, wherein the subject is a mammal.

Embodiment 41: The method of embodiment 40, wherein the mammal is a human.

Embodiment 42: The method of embodiment 40, wherein the mammal is selected from a canine, cat, ferret, rabbit, cattle, horse, monkey, and coyote.

Embodiment 43: Use of a recombinant rabies virus glycoprotein of any one of embodiments 1-9 or a glycoprotein trimer of embodiment 10 for the manufacture of a medicament for the treatment in a subject of a viral disease.

Embodiment 44: A recombinant rabies virus glycoprotein of any one of embodiments—1-9 or a glycoprotein trimer of embodiment 10 for use in the treatment of a viral disease in a subject in need thereof.

### Examples

The recombinant rabies virus glycoprotein provided herein including embodiments thereof include a rabies virus glycoprotein and, optionally, a trimerization domain. The trimeric rabies virus glycoprotein of the present application has a series of point mutations to the protein in order to stabilize it in its trimeric, pre-fusion conformation. The mutations consist of changes to four residues on the trimeric interface of RabvG: His20Met, His261Gln, Tyr77Ala, and Ser289Leu (FIG. 1).

Referring to the results in Table 1 and FIG. 2, these mutations increase the proportion of soluble RabvG in the pre-fusion conformation by ˜700% compared to the wild-type glycoprotein (Table 1) and inhibit the transition from pre-fusion to post-fusion in the full-length glycoprotein (FIG. 2), indicating increased protein stability.

TABLE 1
20M, 261Q, 289L, 261Q/289L, and 20M/261Q/289L mutations increase
the proportion of pre-fusion RabvG for the soluble ectodomain.

| | | Proportion of |
|---|---|---|
| Mutant | Secretion | pre-fusion RabvG |
| Wild-type RabvG | 1.00 | 1.00 |
| 20M RabvG | 0.89 | 1.18 |
| 261Q RabvG | 0.66 | 1.30 |
| 289L RabvG | 0.65 | 2.45 |
| 261Q/289L RabvG | 0.53 | 3.42 |
| 20M/261Q/289L RabvG | 0.33 | 6.83 |

Plasmids encoding soluble RabvG ectodomains were transfected into 293T cells. 4 days post transfection, tissue culture supernatant was collected. Total amount of protein secreted into supernatant (Secretion) and the proportion of secreted glycoprotein in the pre-fusion conformation (Proportion of pre-fusion RabvG) was measured via ELISA using a polyclonal anti-rabies G antibody and a monoclonal, pre-fusion conformation specific RabvG antibody, respectively. Secretion and proportion of pre-fusion RabvG were normalized to wild-type, PV strain RabvG. The results indicate that although the 20M/261Q/289L combination mutant had less total protein secreted into the supernatant, what was secreted was ˜7 times more likely to be in the pre-fusion conformation than wild-type RabvG.

As illustrated in FIG. 2, the stabilizing mutations of 289L and 20M/261Q/289L decrease the pH at which RabvG fuses cells. 293T cells constitutively expressing eGFP or mCherry were seeded together in 24 well plates and transfected to express full-length RabvG. 2 days post transfection, cells were incubated in 0.1M citric acid/trisodium citrate buffer at the indicated pH (FIG. 2) or left in DMEM growth medium (No Change) for 10 minutes at room temperature, then washed with PBS and incubated with DMEM for 1 hr at 37° C. Cells were fixed and imaged to assess fusion (cells were stained to measure cell fusion, where in the experiment the color indicated cell fusion.) Based on the imaging results, these results indicated that wild-type RabvG fused cells at pH 5.83. The 289L mutant fused cells at pH 5.17 and the 20M/261Q/289L mutant did not fuse at any of the tested pHs. Accordingly, these results demonstrate that the 289L mutant increases protein stability and the 20M/261Q/289L mutant greatly increases protein stability.

Table 1 illustrates that the 20Met/261Gln/289Leu mutation reduces secretion of the soluble RabvG ectodomain into supernatant. FIG. 3 illustrates the experiment where 293T cells were transfected with full-length RabvG carrying the indicated point mutations. Four days post-transfection, tissue culture supernatant was collected and an ELISA was performed to measure the amount of RabvG cleaved from the cell surface and released into the supernatant. RabvG containing the point mutations 20M, 261Q, and 289L singly or in combination was not cleaved and released into tissue culture supernatant to significant levels.

Turning to FIG. 4, 293T cells were transfected to express full-length RabvG with the indicated point mutations. Two days post transfection, cells were fixed and stained for analysis of surface-expressed RabvG via flow cytometry and indirect immunofluorescence assay (IFA). For the flow cytometry experiment in FIG. 4A, cells were gated on recognition by a polyclonal anti-RabvG antibody to detect transfected cells, and then recognition of these cells by a pre-fusion conformation specific monoclonal antibody was measured. The level of surface expressed RabvG was not significantly different between the wild-type protein or any of the point mutants therefore 20M, 261Q, 289L, and 20M/261Q/289L mutant full-length RabvG reached the cell surface to the same levels as wild-type RabvG.

Further, 77Ala aids in the purifying of stabilize glycoprotein ectodomain, resulting in a formulation better suited for structural biology or, as embodiments of the present disclosure set forth, a protein subunit vaccine.

In FIG. 16, 293T cells constitutively expressing half of a split GFP molecule were seeded together in 24-well plates and transfected to express full-length RABV-G. Two days post transfection, cells were incubated in 0.1M citric acid/trisodium citrate buffer at the indicated pH for 10 minutes at room temperature, then washed with PBS and incubated with DMEM for 1 h at 37° C. Cells were fixed and imaged (via staining) to assess fusion. Brighter cells (via imaging) indicate the formation of syncytia (cell fusion). Wild-type RABV-G induced cell-cell fusion at pH 5.83 and below. Individual stabilizing mutations required decreased pH to trigger fusion, while combinations of two or more point mutations did not induce cell-cell fusion.

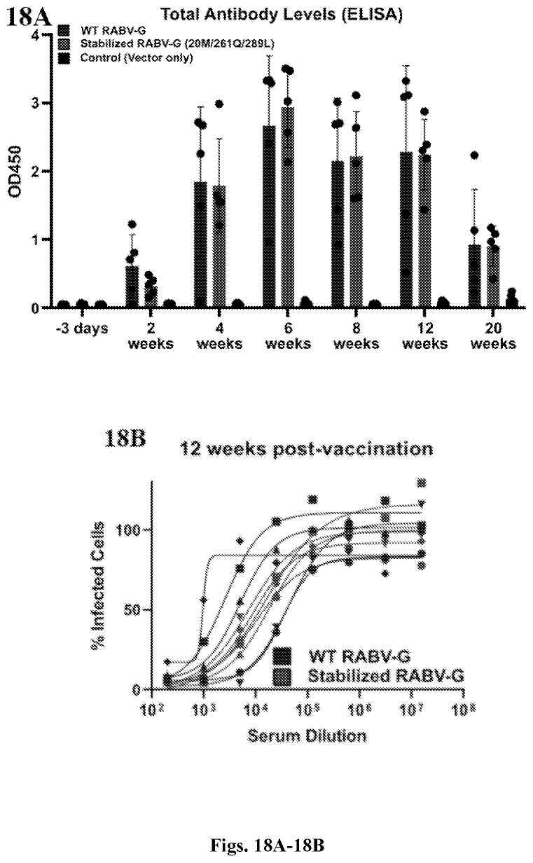

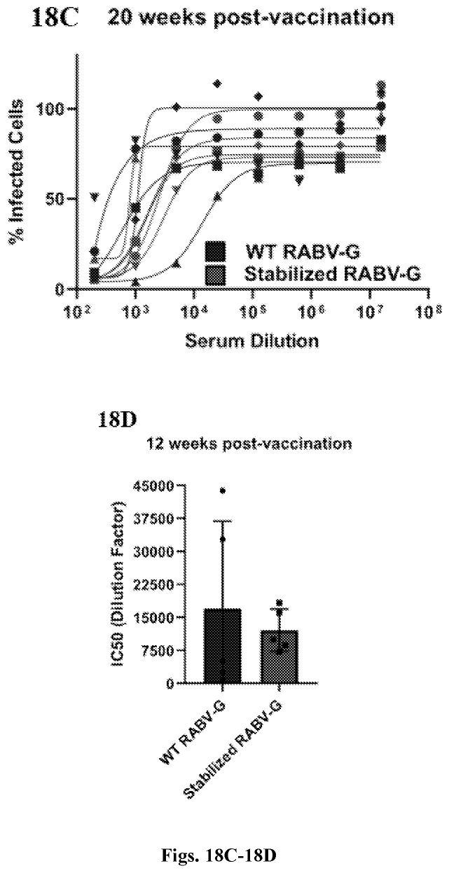

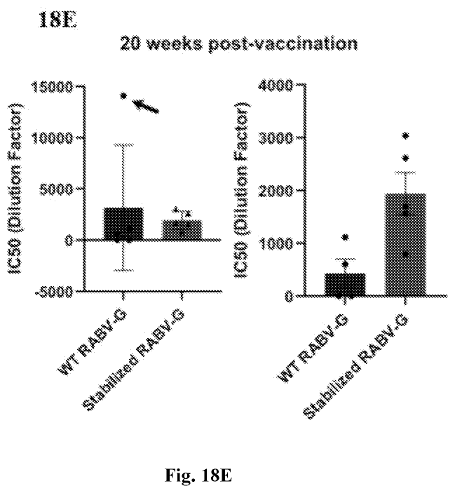

In FIG. 18, antibody titer levels and immune longevity was assessed for mice vaccinated with wild-type and stabilized (20M/261Q/289L) RABV-G. In FIG. 18A, an ELISA of serum from mice immunized with full-length RABV-G plasmids encoding WT or stabilized RABV-G was measured, illustrating antibody binding to wild-type RABV-G ectodomains at the indicated timepoint post-vaccination. FIG. 18B illustrates the neutralization data for mouse serum collected at 12 weeks post-vaccination and FIG. 18D illustrates the IC₅₀ values at 12 weeks post-vaccination. FIG. 18C illustrates the neutralization data for mouse serum collected at 20 weeks post-vaccination and FIG. 18E illustrates the IC₅₀ values at 20-weeks post-vaccination. For the 20-week post-vaccination timepoint, serum from all mice is shown with an outlier indicated by an arrow (left), and serum from all mice minus the indicated outlier is graphed separately (right). Error bars indicate ±SEM.

Accordingly, these results confirm that the recombinant rabies virus glycoprotein with at least one of the mutations of the present application make it an ideal candidate for an RNA vaccine encoding full-length glycoprotein.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

INFORMAL SEQUENCE LISTING

SEQ ID NO: 1 PV Strain RabvG|20M, 261Q, 289L
KFPIYTIPDKLGPWSPIDIMHLSCPNNLVVEDEGCTNLSGFSYMELKVGYISAIKMNGFTC
TGVVTEAETYTNFVGYVTTTFKRKHFRPTPDACRAAYNWKMAGDPRYEESLHNPYPD
YHWLRTVKTTKESLVIISPSVADLDPYDRSLHSRVFPGGNCSGVAVSSTYCSTNHDYTIW
MPENPRLGMSCDIFTNSRGKRASKGSETCGFVDERGLYKSLKGACKLKLCGVLGLRLM
DGTWVAMQTSNETKWCPPGQLVNLQDFRSDEIEHLVVEELVKKREECLDALELIMTTK
SVSFRRLSHLRKLVPGFGKAYTIFNKTLMEADAHYKSVRTWNEIIPSKGCLRVGGRCHP
HVNGVFFNGIILGPDGNVLIPEMQSSLLQQHMELLVSSVIPLMHPLADPSTVFKNGDEAE
DFVEVHL
SEQ ID NO: 2 PV Strain RabvG|20M, 77A, 261Q, 289L
KFPIYTIPDKLGPWSPIDIMHLSCPNNLVVEDEGCTNLSGFSYMELKVGYISAIKMNGFTC
TGVVTEAETYTNFVGAVTTTFKRKHFRPTPDACRAAYNWKMAGDPRYEESLHNPYPD
YHWLRTVKTTKESLVIISPSVADLDPYDRSLHSRVFPGGNCSGVAVSSTYCSTNHDYTIW
MPENPRLGMSCDIFTNSRGKRASKGSETCGFVDERGLYKSLKGACKLKLCGVLGLRLM
DGTWVAMQTSNETKWCPPGQLVNLQDFRSDEIEHLVVEELVKKREECLDALELIMTTK
SVSFRRLSHLRKLVPGFGKAYTIFNKTLMEADAHYKSVRTWNEIIPSKGCLRVGGRCHP
HVNGVFFNGIILGPDGNVLIPEMQSSLLQQHMELLVSSVIPLMHPLADPSTVFKNGDEAE
DFVEVHL
SEQ ID NO: 3 Wild-type RabvG
KFPIYTIPDKLGPWSPIDIHHLSCPNNLVVEDEGCTNLSGFSYMELKVGYISAIKMNGFTC
TGVVTEAETYTNFVGYVTTTFKRKHFRPTPDACRAAYNWKMAGDPRYEESLHNPYPD
YHWLRTVKTTKESLVIISPSVADLDPYDRSLHSRVFPGGNCSGVAVSSTYCSTNHDYTIW
MPENPRLGMSCDIFTNSRGKRASKGSETCGFVDERGLYKSLKGACKLKLCGVLGLRLM
DGTWVAMQTSNETKWCPPGQLVNLHDFRSDEIEHLVVEELVKKREECLDALESIMTTK
SVSFRRLSHLRKLVPGFGKAYTIFNKTLMEADAHYKSVRTWNEIIPSKGCLRVGGRCHP
HVNGVFFNGIILGPDGNVLIPEMQSSLLQQHMELLVSSVIPLMHPLADPSTVFKNGDEAE
DFVEVHL

What is claimed is:

1. A recombinant rabies virus glycoprotein comprising a rabies virus glycoprotein comprising one or more of the following mutations: His20Met, His261Gln, Tyr77Ala, and Ser289Leu.

2. The recombinant rabies virus glycoprotein of claim 1, wherein the mutation is at least His20Met.

3. The recombinant rabies virus glycoprotein of claim 1, wherein the mutation is at least His261Gln

4. The recombinant rabies virus glycoprotein of claim 1, wherein the mutation is at least Tyr77Ala.

5. The recombinant rabies virus glycoprotein of claim 1, wherein the mutation is at least Ser289Leu.

6. A recombinant rabies virus glycoprotein comprising a rabies virus glycoprotein having an amino acid sequence of SEQ ID NO:1 or SEQ ID NO:2.

7. The recombinant rabies virus glycoprotein of claim 6, wherein the rabies virus glycoprotein comprises an amino acid sequence as set forth in SEQ ID NO:1.

8. The recombinant rabies virus glycoprotein of claim 6, wherein the rabies virus glycoprotein comprises an amino acid sequence as set forth in SEQ ID NO:2.

9. A recombinant rabies virus glycoprotein comprising a rabies virus glycoprotein having an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:1 or SEQ ID NO:2.

10. A glycoprotein trimer comprising the recombinant rabies virus glycoprotein of claim 1.

11. A nucleic acid encoding the recombinant rabies virus glycoprotein of claim 1 or the glycoprotein trimer of claim 10.

12. The nucleic acid of claim 11 further comprising a vector.

13. A cell comprising the recombinant rabies virus glycoprotein of claim 1 or the glycoprotein trimer of claim 10.

14. A cell comprising the nucleic acid of claim 11.

15. The cell of claim 13, wherein the cell is a human cell.

16. A vaccine composition comprising the recombinant rabies virus glycoprotein of claim 1 and a pharmaceutically acceptable excipient.

17. A vaccine composition comprising the glycoprotein trimer of claim 10 and a pharmaceutically acceptable excipient.

18. The vaccine composition of claim 17 further comprising an adjuvant.

19. A method of treating or preventing a viral disease in a subject in need of such treatment or prevention, said method comprising administering a therapeutically or prophylactically effective amount of the recombinant rabies virus glycoprotein of claim 1 to the subject.

20. A method of treating or preventing a viral disease in a subject in need of such treatment or prevention, said method comprising administering a therapeutically or prophylactically effective amount of the glycoprotein trimer of claim 10 to the subject.

21. The method of claim 19, wherein said viral disease is caused by a member of the Rhabdoviridae family.

22. The method of claim 21, wherein said viral disease is a lyssavirus.

23. The method of claim 22, wherein said viral disease is a rabies virus.

24. The method of claim 19, wherein the subject is a mammal.

25. The method of claim 24, wherein the mammal is human.

26. The method of claim 24, wherein the mammal is selected from a canine, cat, ferret, rabbit, cattle, horse, monkey, and coyote.

27. A method for immunizing a subject susceptible to a viral disease, comprising administering the recombinant rabies virus glycoprotein of claim 1 to a subject under conditions such that antibodies directed to said rabies virus glycoprotein or a fragment thereof are produced.

28. The method of claim 27, wherein the subject is a mammal.

29. The method of claim 28, wherein the mammal is a human.

30. The method of claim 28, wherein the mammal is selected from a canine, cat, ferret, rabbit, cattle, horse, monkey, and coyote.

31. A method for immunizing a subject susceptible to a viral disease, comprising administering the glycoprotein trimer of claim 10 to a subject under conditions such that antibodies directed to said glycoprotein trimer or a fragment thereof are produced.

32. The method of claim 31, wherein the subject is a mammal.

33. The method of claim 32, wherein the mammal is a human.

34. The method of claim 32, wherein the mammal is selected from a canine, cat, ferret, rabbit, cattle, horse, monkey, and coyote.

35. A method of diagnosing rabies virus infection in a subject, the method comprising: (a) contacting a biological sample obtained from the subject with the recombinant rabies virus glycoprotein of claim 1, and (b) detecting binding of one or more antibodies to said recombinant rabies virus glycoprotein, thereby diagnosing rabies virus infection in said subject.

36. The method of claim 35, wherein the subject is a mammal.

37. The method of claim 36, wherein the mammal is a human.

38. The method of claim 36, wherein the mammal is selected from a canine, cat, ferret, rabbit, cattle, horse, monkey, and coyote.

39. A method of diagnosing rabies virus infection in a subject, the method comprising: (a) contacting a biological sample obtained from the subject with the glycoprotein trimer of claim 10, and (b) detecting binding of one or more antibodies to said glycoprotein trimer, thereby diagnosing rabies virus infection in said subject.

40. The method of claim 39, wherein the subject is a mammal.

41. The method of claim 40, wherein the mammal is a human.

42. The method of claim 40, wherein the mammal is selected from a canine, cat, ferret, rabbit, cattle, horse, monkey, and coyote.

43. Use of a recombinant rabies virus glycoprotein of claim 1 or a glycoprotein trimer of claim 10 for the manufacture of a medicament for the treatment in a subject of a viral disease.

44. A recombinant rabies virus glycoprotein of claim 1 or a glycoprotein trimer of claim 10 for use in the treatment of a viral disease in a subject in need thereof.
